(19) United States
(12) Patent Application Publication
(10) Pub. No.: US 2014/0364445 A1
(43) Pub. Date: Dec. 11, 2014

(54) PHARMACEUTICAL COMPOSITION COMPRISING A TRPA1 ANTAGONIST AND AN ANTICHOLINERGIC AGENT

(71) Applicant: GLENMARK PHARMACEUTICALS S.A., La Chaux-de-Fonds (CH)

(72) Inventors: Neelima Khairatkar-Joshi, Thane (IN); Raghuram Anupindi, Navi Mumbai (IN); Selvan Vaiyapuri Thamil, Trichy (IN); Abhay Kulkarni, Navi Mumbai (IN); Amol Waghchoure, Navi Mumbai (IN)

(21) Appl. No.: 14/362,003

(22) PCT Filed: Dec. 5, 2012
(86) PCT No.: PCT/IB2012/056966

§ 371 Date: May 30, 2014

(30) Foreign Application Priority Data
Dec. 5, 2011 (IN) 3418/MUM/2011

(51) Int. Cl. A61K 31/519; A61K 31/439

(52) U.S. Cl. CPC A61K 31/519; A61K 31/439
USPC 514/260.1

(57) ABSTRACT

The present invention relates to a pharmaceutical composition comprising a transient receptor potential ankyrin-1 receptor (“TRPA1”) antagonist and an anticholinergic agent. Particularly, the present invention provides a pharmaceutical composition comprising a TRPA1 antagonist having IC₅₀ for inhibiting human TRPA1 receptor activity of less than 1 micromolar and an anticholinergic agent; a process for preparing such composition; and its use in treating a respiratory disorder in a subject.

### PRIORITY DOCUMENT

This patent application claims priority to Indian Provisional Patent Application number 3418/MUM/2011 (filed on Dec. 5, 2011), the contents of which are incorporated by reference herein.

### FIELD OF THE INVENTION

The present patent application relates to a pharmaceutical composition comprising a transient receptor potential ankyrin-1 receptor (“TRPA1”) antagonist and an anticholinergic agent. Particularly, the application provides a pharmaceutical composition comprising a TRPA1 antagonist having IC₅₀ for inhibiting human TRPA1 receptor activity of less than 1 micromolar and an anticholinergic agent; a process for preparing such composition; and its use in treating a respiratory disorder in a subject.

### BACKGROUND OF THE INVENTION

Respiratory disorders related to airway inflammation include a number of severe lung diseases including asthma and chronic obstructive pulmonary disease (“COPD”). The airways of asthmatic patients are infiltrated by inflammatory leukocytes, of which the eosinophil is believed to be the most prominent component. Inflammatory sensitization of airway neurons is believed to increase nasal and cough sensitivity, heighten the sense of irritation, and promote fluid secretion, airway narrowing, and bronchoconstriction.

TRPA1 receptor activation in the airways by exogenous noxious stimuli, including cold temperatures (generally, less than about 17° C.), pungent natural compounds (e.g., mustard, cinnamon and garlic), tobacco smoke, tear gas and environmental irritants as well as by endogenous biochemical mediators released during inflammation, is supposed to be one of the mechanisms for neurogenic inflammation in the airways. Neurogenic inflammation is an important component of chronic airway diseases like COPD and asthma.

International Publication Nos. viz., WO 2004/055054, WO 2005/089206, WO 2007/073505, WO 2008/0949099, WO 2009/089082, WO 2009/002933 WO 2009/158719, WO 2009/144548, WO 2010/004390, WO 2010/109287, WO 2010/109334, WO 2010/109329, WO 2010/109328, WO 2010/125469 and WO 2010/004390 describe various transient receptor potential (“TRP”) receptor modulators.

Anticholinergic agents are believed to inhibit vagally-mediated reflexes by blocking acetylcholine at the cholinergic receptor. Anticholinergic agents are also believed to inhibit secretions of the serous and sero-mucous glands of the nasal mucosa. Anticholinergic agents for treatment or control of respiratory disorders include tiotropium, oxitropium, ipratropium, glycopyrrolate and aclidinium or salt thereof.

One known anticholinergic agent is tiotropium bromide, the chemical name of which is, (1α, 2β, 4β, 5α, 7β)-7-[(hydroxydi-2-thienylacetyl)oxy]-9,9-dimethyl-3-oxa-9-azoniatricyclo[3.3.1.02,4] nonane bromide monohydrate. Tiotropium bromide is available commercially as SPIRIVA® capsules containing 18 mcg tiotropium (equivalent to 22.5 mcg tiotropium bromide monohydrate) marketed by Boehringer Ingelheim Pharmaceuticals, Inc. in the United States. Tiotropium bromide is indicated for the maintenance treatment of bronchospasm associated with COPD, and for reducing COPD exacerbations.

Another anticholinergic agent, ipratropium bromide is chemically, 8-azoniabicyclo (3.2.1) octane, 3-(3-hydroxy-1-oxo-2-phenylpropoxy)-8-methyl-8-(1-methylethyl)-, bromide monohydrate. Ipratropium bromide is commercially available as ATROVENT® 0.06% nasal spray (42 mcg per spray) marketed by Boehringer Ingelheim Pharmaceuticals, Inc. in the United States. It is administered as a pressurized metered-dose aerosol unit for oral inhalation. Each actuation of the inhaler delivers 21 mcg of ipratropium bromide. Ipratropium bromide is indicated for the symptomatic relief of rhinorrhea associated with the common cold or seasonal allergic rhinitis. ATROVENT HFA® is another product of ipratropium bromide is approved as a bronchodilator for maintenance treatment of bronchospasm associated with chronic obstructive pulmonary disease (COPD), including chronic bronchitis and emphysema.

Aclidinium bromide, chemically known as [(8R)-1-(3-phenoxypropyl)-1-azoniabicyclo[2.2.2]octan-8-yl]2-hydroxy-2,2-dithiophen-2-ylacetate bromide is a novel long acting inhaled muscarinic antagonist. It is approved as TUDORZA® PRESSAIR® 0.375 mg/INH in the United States. It is approved for the long-term maintenance treatment of bronchospasm associated with chronic obstructive pulmonary disease (COPD), including chronic bronchitis and emphysema.

Another anticholinergic agent, glycopyrrolate is chemically 3-(2 cyclopentyl-2-hydroxy-2-phenylacetoxy)-1,1-dimethylpyrrolidinium

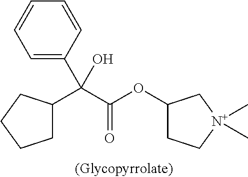 [SMILES: C[N+]1(C)CCC(OC(=O)C(O)(c2ccccc2)C2CCCC2)C1]

Glycopyrrolate is being developed for the treatment of asthma and COPD.

There still exists a need for an effective therapeutic treatment for respiratory disorders like asthma, COPD and rhinorrhea.

### SUMMARY OF THE INVENTION

The present invention relates to a pharmaceutical composition comprising a TRPA1 antagonist and an anticholinergic agent.

The inventors have surprisingly found that a TRPA1 antagonist and an anticholinergic agent act synergistically in the treatment of respiratory disorders and are more effective and provide better therapeutic value than treatment with either active ingredient alone.

The anticholinergic agent, as contemplated herein, including tiotropium, oxitropium, ipratropium, glycopyrrolate and aclidinium or their salt may be present in the form of their stereoisomers, polymorphs, and solvates, including hydrates, all of which are included in the scope of the invention.

In another embodiment, the present invention relates to a pharmaceutical composition comprising synergistically effective amount of a TRPA1 antagonist having an IC₅₀ for inhibiting human TRPA1 receptor activity of less than 1 micromolar having structure of formulae: (XII) or (D)

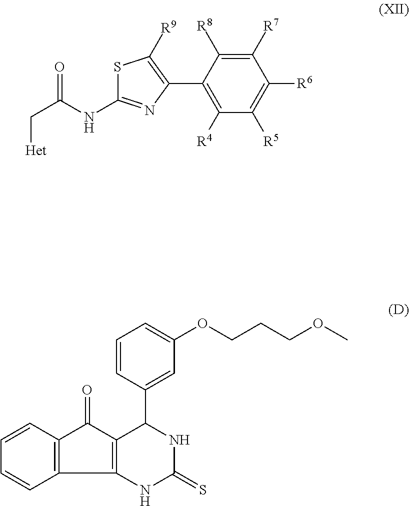

or a pharmaceutically-acceptable salt thereof, wherein, ‘Het’ is selected from the group consisting of

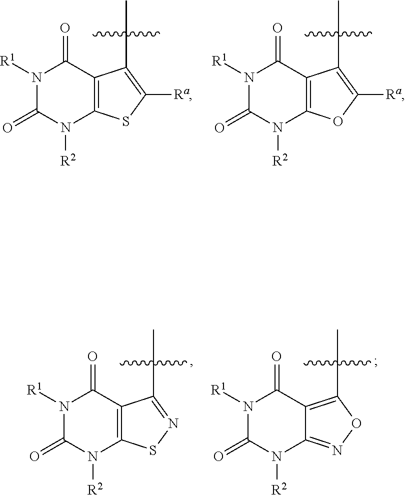

R¹, R² and R^{a}, which may be the same or different, are each independently hydrogen or (C₁-C₄) alkyl;
R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl and an anticholinergic agent.

In an aspect of the embodiment, the TRPA1 antagonist as contemplated herein and the anticholinergic agent are present in a weight ratio ranging from about 1:0.0001 to about 1:10000.

In yet another embodiment, the present invention relates to a pharmaceutical composition comprising synergistically effective amount of a TRPA1 antagonist having structure of formula:

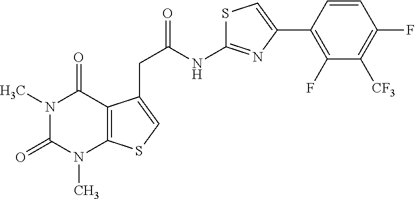 [SMILES: Cn1c(=O)c2c(CC(=O)Nc3nc(-c4ccc(F)c(C(F)(F)F)c4F)cs3)csc2n(C)c1=O]

and an anticholinergic agent.

In an embodiment, the present invention relates to a method of treating a respiratory disorder in a subject, said method comprising administering to the subject the pharmaceutical composition comprising synergistically effective amount of a TRPA1 antagonist having an IC₅₀ for inhibiting human TRPA1 receptor activity of less than 1 micromolar as contemplated herein and an anticholinergic agent.

The respiratory disorder, in the context of present invention, includes but is not limited to airway inflammation, asthma, emphysema, bronchitis, COPD, sinusitis, rhinitis, cough, respiratory depression, reactive airways dysfunction syndrome (RADS), acute respiratory distress syndrome (ARDS), irritant induced asthma, occupational asthma, sensory hyper-reactivity, multiple chemical sensitivity, and aid in smoking cessation therapy.

In a further embodiment, the present invention relates to a method of treating a respiratory disorder in a subject, said method comprising administering the subject a pharmaceutical composition comprising synergistically effective amount of a TRPA1 antagonist having an IC₅₀ for inhibiting human TRPA1 receptor activity of less than 1 micromolar as contemplated herein and an anticholinergic agent selected from a group consisting of tiotropium, oxitropium, ipratropium, glycopyrrolate and aclidinium or salts thereof.

In a further embodiment, the present invention relates to use of synergistically effective amount of a TRPA1 antagonist having an IC₅₀ for inhibiting human TRPA1 receptor activity of less than 1 micromolar as contemplated herein and an anticholinergic agent in the preparation of a pharmaceutical composition of the present invention for the treatment of a respiratory disorder in a subject.

In a further embodiment, the present invention relates to a pharmaceutical composition comprising synergistically effective amount of a TRPA1 antagonist having an IC₅₀ for inhibiting human TRPA1 receptor activity of less than 1 micromolar as contemplated herein and an anticholinergic agent for the treatment of a respiratory disorder in a subject.

In an embodiment, the present invention relates to a pharmaceutical composition comprising synergistically effective amount of a TRPA1 antagonist having structure of formula:

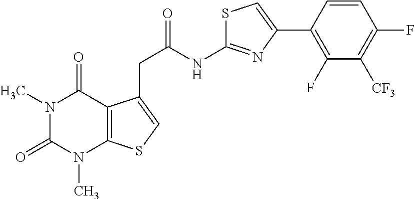 [SMILES: Cn1c(=O)c2c(CC(=O)Nc3nc(-c4ccc(F)c(C(F)(F)F)c4F)cs3)csc2n(C)c1=O]

and an anticholinergic agent selected from a group consisting of tiotropium, oxitropium, ipratropium, glycopyrrolate and aclidinium or salts thereof. In an aspect of this embodiment, the pharmaceutical composition is a fixed dose combination. In another aspect of the embodiment, the composition is for inhalation administration.

In yet another aspect of this embodiment, the composition is for inhalation administration, wherein the TRPA1 antagonist and the anticholinergic agent are present in a weight ratio from about 1:0.001 to about 1:300.

In an embodiment, the present invention relates to a method of treating a respiratory disorder in a subject, said method comprising administering to the subject the pharmaceutical composition comprising synergistically effective amount of a TRPA1 antagonist having structure of formula:

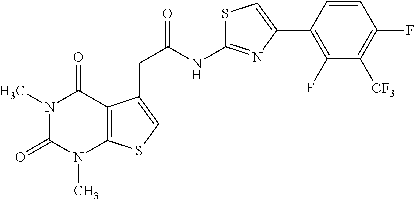 [SMILES: Cn1c(=O)c2c(CC(=O)Nc3nc(-c4ccc(F)c(C(F)(F)F)c4F)cs3)csc2n(C)c1=O]

and an anticholinergic agent. In an aspect of this embodiment, the anticholinergic agent selected from a group consisting of tiotropium, oxitropium, ipratropium, glycopyrrolate and aclidinium or salts thereof. In another aspect of the embodiment, the composition is for inhalation administration.

In an embodiment, the present invention relates to a method of treating COPD by reducing neutrophil count in a subject, said method comprising administering to the subject the pharmaceutical composition comprising synergistically effective amount of a TRPA1 antagonist having structure of formula:

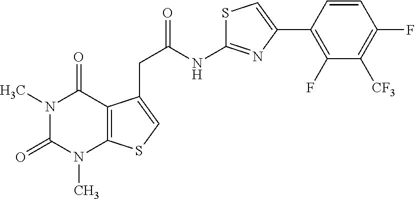 [SMILES: Cn1c(=O)c2c(CC(=O)Nc3nc(-c4ccc(F)c(C(F)(F)F)c4F)cs3)csc2n(C)c1=O]

and an anticholinergic agent. In an aspect of this embodiment, the anticholinergic agent selected from a group consisting of tiotropium, oxitropium, ipratropium, glycopyrrolate and aclidinium or salts thereof. In another aspect of this embodiment, the respiratory disorder is asthma. In another aspect of the embodiment, the composition is for inhalation administration.

In an embodiment, the present invention relates to a method of reducing neutrophil count in a subject, said method comprising administering to the subject the pharmaceutical composition comprising synergistically effective amount of a TRPA1 antagonist having structure of formula:

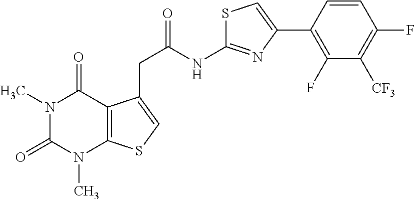 [SMILES: Cn1c(=O)c2c(CC(=O)Nc3nc(-c4ccc(F)c(C(F)(F)F)c4F)cs3)csc2n(C)c1=O]

and an anticholinergic agent. In an aspect of this embodiment, the anticholinergic agent selected from a group consisting of tiotropium, oxitropium, ipratropium, glycopyrrolate and aclidinium or salts thereof. In another aspect of the embodiment, the composition is for inhalation administration.

In an embodiment, the present invention relates to a method of treating asthma by inhibiting airway resistance in a subject, said method comprising administering to the subject the pharmaceutical composition comprising synergistically effective amount of a TRPA1 antagonist having structure of formula:

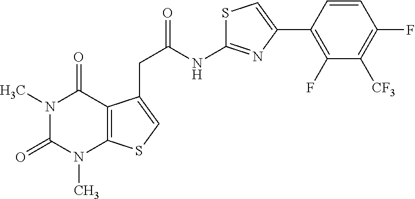 [SMILES: Cn1c(=O)c2c(CC(=O)Nc3nc(-c4ccc(F)c(C(F)(F)F)c4F)cs3)csc2n(C)c1=O]

and an anticholinergic agent. In an aspect of this embodiment, the anticholinergic agent selected from a group consisting of tiotropium, oxitropium, ipratropium, glycopyrrolate and aclidinium or salts thereof. In another aspect of this embodiment, the respiratory disorder is asthma. In another aspect of the embodiment, the composition is for inhalation administration.

In an embodiment, the present invention relates to a method of inhibiting airway resistance in a subject, said method comprising administering to the subject the pharmaceutical composition comprising synergistically effective amount of a TRPA1 antagonist having structure of formula:

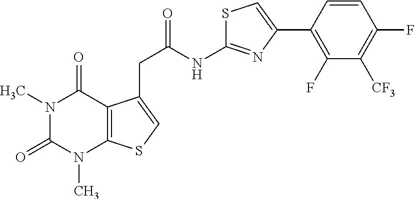 [SMILES: Cn1c(=O)c2c(CC(=O)Nc3nc(-c4ccc(F)c(C(F)(F)F)c4F)cs3)csc2n(C)c1=O]

and an anticholinergic agent. In an aspect of this embodiment, the anticholinergic agent selected from a group consisting of tiotropium, oxitropium, ipratropium, glycopyrrolate and aclidinium or salts thereof. In another aspect of the embodiment, the composition is for inhalation administration.

In another embodiment, the present invention relates to use of synergistically effective amount of a TRPA1 antagonist having structure of formula:

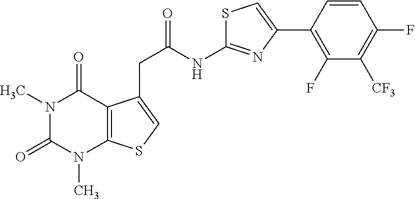 [SMILES: Cn1c(=O)c2c(CC(=O)Nc3nc(-c4ccc(F)c(C(F)(F)F)c4F)cs3)csc2n(C)c1=O]

and an anticholinergic agent in the preparation of a pharmaceutical composition of the present invention for the treatment of a respiratory disorder in a subject. In an aspect of this embodiment, the anticholinergic agent selected from a group consisting of tiotropium, oxitropium, ipratropium, glycopyrrolate and aclidinium or salts thereof. In another aspect of the embodiment, the composition is for inhalation administration.

In a further embodiment, the present invention relates to a pharmaceutical composition comprising synergistically effective amount of a TRPA1 antagonist having structure of formula:

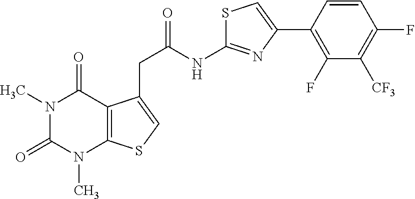 [SMILES: Cn1c(=O)c2c(CC(=O)Nc3nc(-c4ccc(F)c(C(F)(F)F)c4F)cs3)csc2n(C)c1=O]

and an anticholinergic agent for the treatment of a respiratory disorder in a subject. In an aspect of the embodiment, the composition is for inhalation administration.

### BRIEF DESCRIPTION OF THE DRAWINGS

FIG. 1 is a bar graph showing the effect of Compound 52, tiotropium bromide, and their combination on methacholine-induced bronchoconstriction in male Dunkin Hartley guinea pigs.

FIG. 2 is a bar graph showing the effect of Compound 52, aclidinium bromide, and their combination on LPS induced neutrophilia in male SD rats.

FIG. 3 is a bar graph showing the effect of Compound 52, tiotropium and their combination on LPS induced neutrophilia in female SD rats.

FIG. 4 is a bar graph showing the effect of Compound 52, ipratropium bromide and their combination on LPS induced neutrophilia in male SD rats.

### DETAILED DESCRIPTION OF THE INVENTION

### Definitions

The terms used herein are defined as follows. If a definition set forth in the present application and a definition set forth earlier in a provisional application from which priority is claimed are in conflict, the definition in the present application shall control the meaning of the terms.

The term “effective amount” or “therapeutically effective amount” denotes an amount of an active ingredient that, when administered to a subject for treating a respiratory disorder, produces an intended therapeutic benefit in a subject.

The therapeutically effective amount of TRPA1 antagonist as described herein ranges from about 0.1 mcg/kg to about 20 mg/kg, and preferably from about 1 mcg/kg to about 15 mg/kg.

The therapeutically effective amount tiotropium to be administered per day ranges from about 5 μg to about 50 μg and preferably from about 10 μg to about 36 μg. Preferably, the discrete dosage strength of tiotropium or its salt is 18 μg.

The therapeutically effective amount of ipratropium to be administered per day ranges from about 0.05 mg to about 10 mg, and preferably from about 0.1 mg to about 1 mg. Preferably, the discrete dosage strengths of ipratropium or its salt are 168 μg or 336 μg or 504 μg or 672 μg.

The therapeutically effective amount of aclidinium to be administered per day ranges from about 0.05 mg to about 10 mg, and preferably from about 0.1 mg to about 1 mg. Preferably, the discrete dosage strengths of aclidinium or its salt are 200 μg or 400 μg or 800 μg.

The therapeutically effective amount of glycopyrrolate to be administered per day ranges from about 0.01 mg to about 10 mg, and preferably from about 0.1 mg to about 1 mg.

The therapeutically effective ranges of actives are given as above, although larger or smaller amount are not excluded if they fall within the scope of the definition of the above paragraphs.

The term “active ingredient” (used interchangeably with “active” or “active substance” or “drug”) as used herein includes a TRPA1 antagonist, an anticholinergic agent or a pharmaceutically acceptable salt thereof. Preferably, the active ingredient includes TRPA1 antagonist having a human IC₅₀ value of less than 1 micromolar, tiotropium, oxitropium, ipratropium, glycopyrrolate and aclidinium or its salt.

The IC₅₀ value is believed to be measure of the effectiveness of a compound in inhibiting biological or biochemical function. This quantitative measure generally indicates molar concentration of a particular compound (or substance) is needed to inhibit a given biological process by half. In other words, it is the half maximal (50%) inhibitory concentration (IC) of the compound. The IC₅₀ of a drug compound (or active substance) can be determined by constructing a concentration-response curve so as to examine the effect of different concentrations of antagonist on reversing agonist activity. IC₅₀ values can be calculated for a given antagonist by determining the concentration needed to inhibit half of the maximum biological response of the agonist. IC₅₀ values can be used to compare the potency of two antagonists.

By “salt” or “pharmaceutically acceptable salt”, it is meant those salts and esters which are, within the scope of sound medical judgment, suitable for use in contact with the tissues of humans and lower animals without undue toxicity, irritation, and allergic response, commensurate with a reasonable benefit to risk ratio, and effective for their intended use. Representative acid additions salts include the hydrochloride, hydrobromide, sulphate, bisulphate, acetate, oxalate, valerate, oleate, palmitate, stearate, laurate, borate, benzoate, lactate, phosphate, tosylate, mesylate, citrate, maleate, fumarate, succinate, tartrate, ascorbate, glucoheptonate, lactobionate, and lauryl sulphate salts. Representative alkali or alkaline earth metal salts include the sodium, calcium, potassium and magnesium salts.

The term “treating” or “treatment” as used herein also covers the prophylaxis, mitigation, prevention, amelioration, or suppression of a disorder modulated by the TRPA1 receptor, or the anticholinergic agent, or by a combination of the two in a subject.

The respiratory disorder includes but is not limited to airway inflammation, asthma, emphysema, bronchitis, COPD, sinusitis, rhinitis, cough, respiratory depression, reactive airways dysfunction syndrome (RADS), acute respiratory distress syndrome (ARDS), irritant induced asthma, occupational asthma, sensory hyper-reactivity, multiple chemical sensitivity, and aid in smoking cessation therapy. Preferably, the respiratory disorder is asthma or COPD.

The term “subject” includes mammals like human and other animals, such as domestic animals (e.g., household pets including cats and dogs) and non-domestic animals (such as wildlife). Preferably, the subject is a human.

By “pharmaceutically acceptable excipients”, it is meant any of the components of a pharmaceutical composition other than the actives and which are approved by regulatory authorities or are generally regarded as safe for human or animal use.

The term “synergistic” or “synergy”, as used herein, refers to a combination exhibiting an effect greater than would be expected from the sum of the effects of the individual components of the combination alone. The term “synergistic” or “synergy” with regard to the combination of a TRPA1 antagonist with an anticholinergic agent which is used in the treatment of a respiratory disorder (for example, in the form of a pharmaceutical composition, a combination product or a kit according to the invention) refers to an efficacy for the treatment of the respiratory disorder that is greater than would be expected from the sum of their individuals effects. The advantages for the synergistic combinations of the present invention include, but are not limited to, lowering the required dose of one or more of the active compounds of the combination, reducing the side effects of one or more of the active compounds of the combination and/or rendering one or more of the active compounds more tolerable to the subject in need of treatment of the respiratory disorder.

### Combinations

The present invention relates to a pharmaceutical composition comprising a TRPA1 antagonist and an anticholinergic agent.

The inventors have surprisingly found that a TRPA1 antagonist and an anticholinergic agent act synergistically in the treatment of respiratory disorders, and are more effective and provide better therapeutic value than treatment with either active ingredient alone.

In an aspect, TRPA1 antagonists useful in the context of the invention, are selected from one of the following formulae: (A) or (B) or (C) or (D)

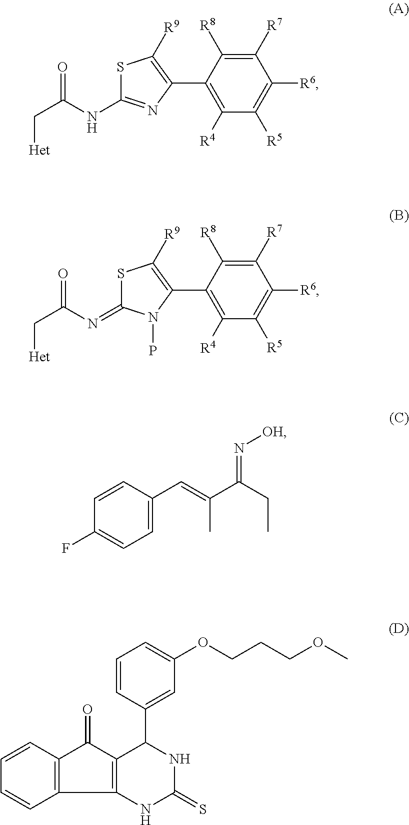

or a pharmaceutically-acceptable salt thereof, wherein, ‘Het’ is selected from the group consisting of

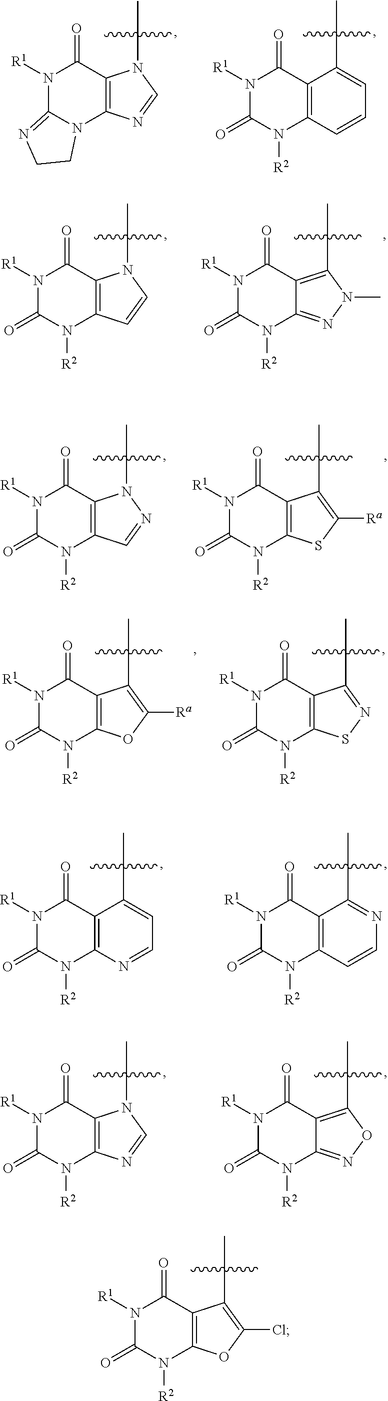

P is selected from

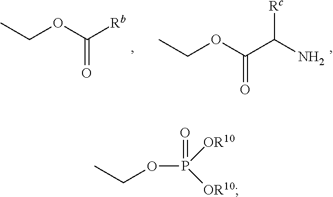

R¹, R² and R^{a}, which may be the same or different, are each independently hydrogen or (C₁-C₄) alkyl;

R^{b} and R^{c} independently selected from hydrogen, substituted or unsubstituted alkyl arylalkyl, amino acid and heterocyclic ring;

R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl;

R¹⁰ is selected from hydrogen, alkyl, arylalkyl and pharmaceutically acceptable cation.

In one aspect, TRPA1 antagonists useful in the context of the invention are selected from those compounds generically or specifically disclosed in WO2009144548. Accordingly, a TRPA1 antagonist useful in the context of the invention has the formula (I):

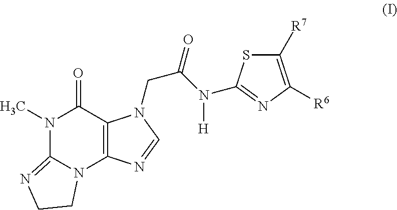

or a pharmaceutically acceptable salt thereof,
wherein,

R⁶ represents hydrogen, substituted or unsubstituted alkyl, substituted or unsubstituted cycloalkyl, substituted or unsubstituted cycloalkylalkyl, substituted or unsubstituted cycloalkenyl, substituted or unsubstituted aryl, substituted or unsubstituted arylalkyl, substituted or unsubstituted heteroaryl, substituted or unsubstituted heteroarylalkyl, substituted or unsubstituted heterocyclic ring and substituted or unsubstituted heterocyclylalkyl;

R⁷ independently represents hydrogen or alkyl.

Few representative TRPA1 antagonists useful in the methods of the invention are mentioned below:

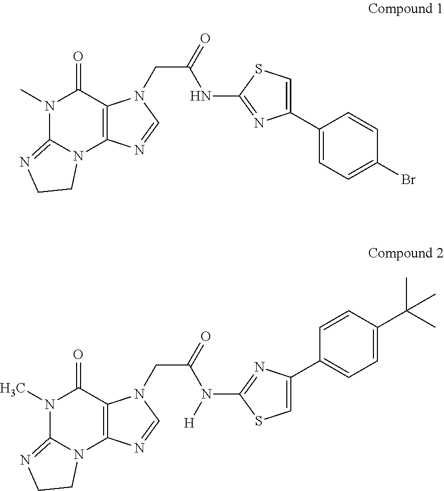 [SMILES: CN1C(=O)c2c(ncn2CC(=O)Nc2nc(-c3ccc(Br)cc3)cs2)N2CCN=C12 ; CN1C(=O)c2c(ncn2CC(=O)Nc2nc(-c3ccc(C(C)(C)C)cc3)cs2)N2CCN=C12]

The preparation of above said compounds is described in WO2009144548.

In another aspect, TRPA1 antagonists useful in the context of the invention are selected from those compounds generically or specifically disclosed in WO2010004390. Accordingly, TRPA1 antagonist useful in the context of the invention has the formula (II):

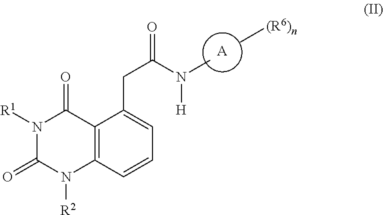

or pharmaceutically acceptable salts thereof,
wherein,

at each occurrence R¹ and R² is independently selected from hydrogen, hydroxyl, substituted or unsubstituted alkyl, substituted or unsubstituted alkenyl, substituted or unsubstituted alkynyl, substituted or unsubstituted cycloalkyl, substituted or unsubstituted cycloalkylalkyl, (CR^{x}R^{y})ₙOR^{x}, COR^{x}, COOR^{x}, CONR^{x}R^{y}, SO₂NR^{x}R^{y}, NR^{x}R^{y}, NR^{x}(CR^{x}R^{y})ₙOR^{x}, NR^{x}(CR^{x}R^{y})ₙCN(CH₂)ₙNR^{x}R^{y}, (CH₂)ₙCHR^{x}R^{y}, (CR^{x}R^{y})NR^{x}R^{y}, NR^{x}(CR^{x}R^{y})ₙCONR^{x}R^{y}, (CH₂)ₙNHCOR^{x} and (CH₂)ₙNH(CH₂)ₙSO₂R^{x}, (CH₂)ₙNHSO₂R^{x};

R^{x} and R^{y} are independently selected from hydrogen, hydroxyl, halogen, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted aryl, substituted or unsubstituted arylalkyl, substituted or unsubstituted heteroaryl, substituted or unsubstituted heteroarylalkyl, substituted or unsubstituted heterocyclic ring and substituted or unsubstituted heterocyclylalkyl;

R^{x} and R^{y} may be joined together to form an optionally substituted 3 to 7 membered saturated, unsaturated or partially saturated cyclic ring, which may optionally include at least two heteroatoms selected from O, NR^{a} or S;

ring A is selected from phenyl, pyridinyl, pyrazolyl, thiazolyl and thiadiazolyl;

each occurrence of R⁶ is independently hydrogen, cyano, nitro, —NR^{x}R^{y}, halogen, hydroxyl, haloalkyl, haloalkoxy, cycloalkylalkoxy, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted aryl, substituted or unsubstituted arylalkyl, substituted or unsubstituted heteroaryl, substituted or unsubstituted heteroarylalkyl, substituted or unsubstituted heterocyclic ring and substituted or unsubstituted heterocyclylalkyl,

R^{x} and R^{y} are independently selected from hydrogen, hydroxyl, halogen, substituted or unsubstituted alkyl, substituted or unsubstituted cycloalkyl, substituted or unsubstituted cycloalkylalkyl, substituted or unsubstituted aryl, substituted or unsubstituted arylalkyl, substituted or unsubstituted heteroaryl, and substituted or unsubstituted heteroarylalkyl;

at each occurrence of ‘n’ is independently selected from 1 to 5.

According to one aspect, specifically provided are compounds of the formula (IIa)

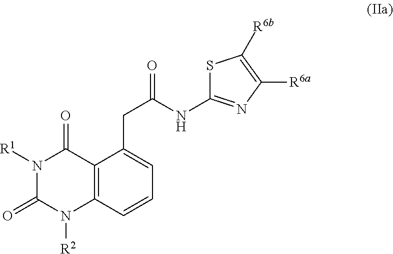

or pharmaceutically acceptable salts thereof,
wherein,

R¹ and R² are as defined above for the compound of formula (II);

R^{6a} and R^{6b} are independently selected from hydrogen, cyano, nitro, —NR^{x}R^{y}, halogen, hydroxyl, haloalkyl, haloalkoxy, cycloalkylalkoxy, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted aryl, substituted or unsubstituted arylalkyl, substituted or unsubstituted heteroaryl, substituted or unsubstituted heteroarylalkyl, substituted or unsubstituted heterocyclic ring and substituted or unsubstituted heterocyclylalkyl, —C(O)OR^{x}, —ORx, —C(O)NR^{x}R^{y}, —C(O)R^{x}, —SO₂R^{x}, —SO₂—NR^{x}R^{y}.

Few representative TRPA1 antagonists useful in the context of the invention are mentioned below:

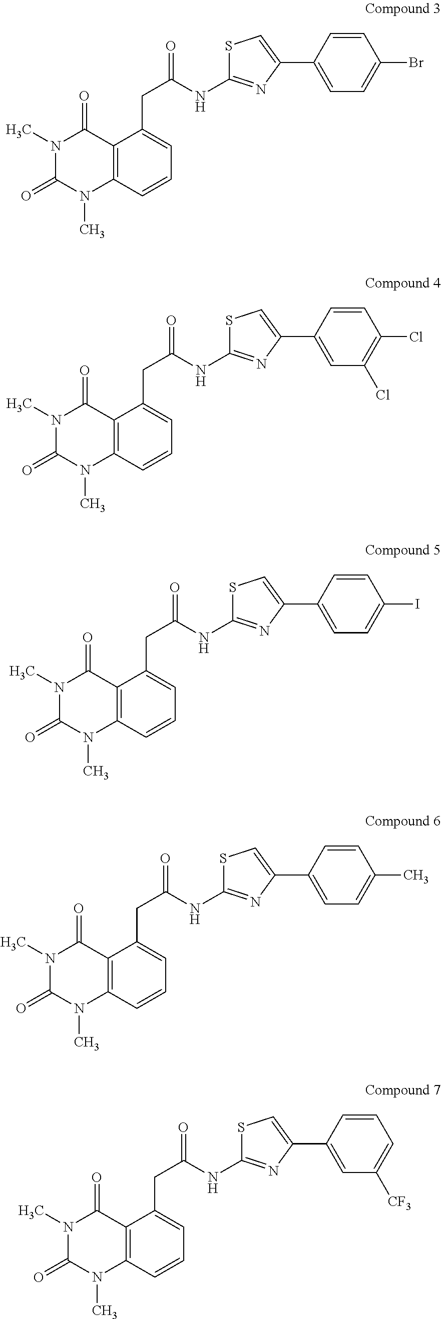 [SMILES: Cn1c(=O)c2c(CC(=O)Nc3nc(-c4ccc(Br)cc4)cs3)cccc2n(C)c1=O ; Cn1c(=O)c2c(CC(=O)Nc3nc(-c4ccc(Cl)c(Cl)c4)cs3)cccc2n(C)c1=O ; Cn1c(=O)c2c(CC(=O)Nc3nc(-c4ccc(I)cc4)cs3)cccc2n(C)c1=O ; Cc1ccc(-c2csc(NC(=O)Cc3cccc4c3c(=O)n(C)c(=O)n4C)n2)cc1 ; Cn1c(=O)c2c(CC(=O)Nc3nc(-c4cccc(C(F)(F)F)c4)cs3)cccc2n(C)c1=O]

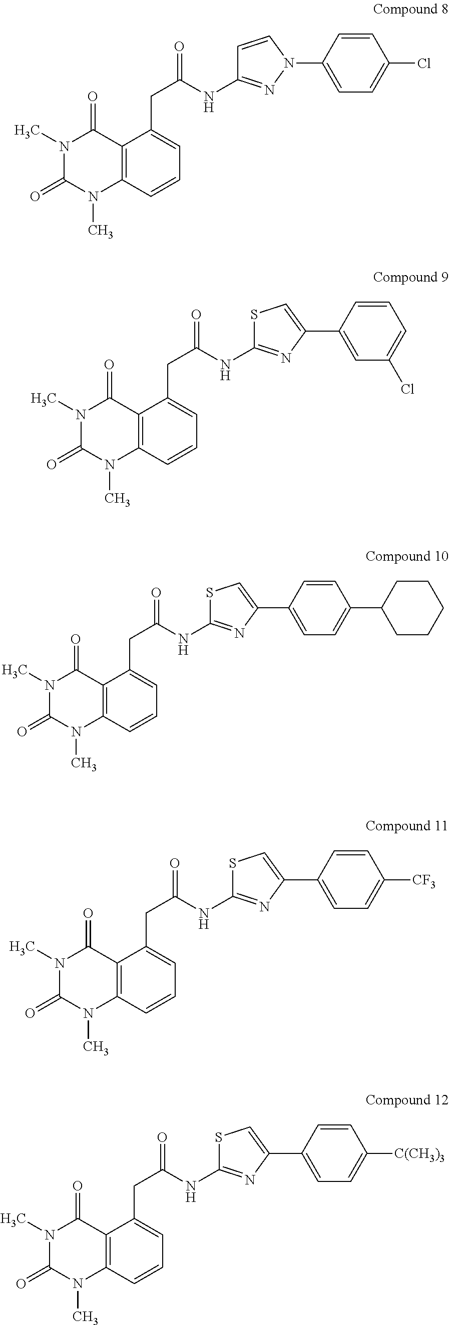 [SMILES: Cn1c(=O)c2c(CC(=O)Nc3ccn(-c4ccc(Cl)cc4)n3)cccc2n(C)c1=O ; Cn1c(=O)c2c(CC(=O)Nc3nc(-c4cccc(Cl)c4)cs3)cccc2n(C)c1=O ; Cn1c(=O)c2c(CC(=O)Nc3nc(-c4ccc(C5CCCCC5)cc4)cs3)cccc2n(C)c1=O ; Cn1c(=O)c2c(CC(=O)Nc3nc(-c4ccc(C(F)(F)F)cc4)cs3)cccc2n(C)c1=O ; Cn1c(=O)c2c(CC(=O)Nc3nc(-c4ccc(C(C)(C)C)cc4)cs3)cccc2n(C)c1=O]

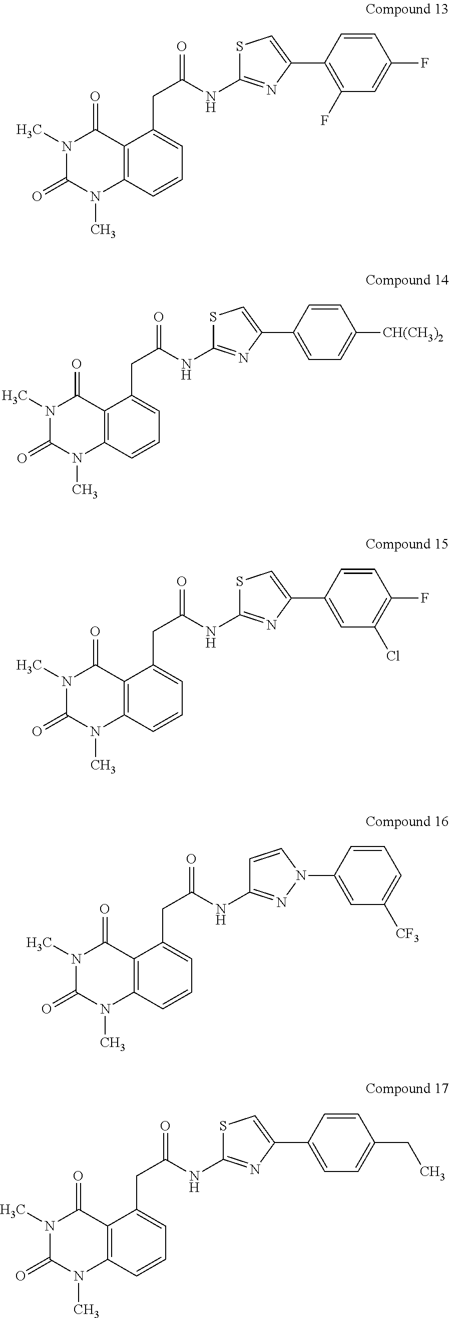 [SMILES: Cn1c(=O)c2c(CC(=O)Nc3nc(-c4ccc(F)cc4F)cs3)cccc2n(C)c1=O ; CC(C)c1ccc(-c2csc(NC(=O)Cc3cccc4c3c(=O)n(C)c(=O)n4C)n2)cc1 ; Cn1c(=O)c2c(CC(=O)Nc3nc(-c4ccc(F)c(Cl)c4)cs3)cccc2n(C)c1=O ; Cn1c(=O)c2c(CC(=O)Nc3ccn(-c4cccc(C(F)(F)F)c4)n3)cccc2n(C)c1=O ; CCc1ccc(-c2csc(NC(=O)Cc3cccc4c3c(=O)n(C)c(=O)n4C)n2)cc1]

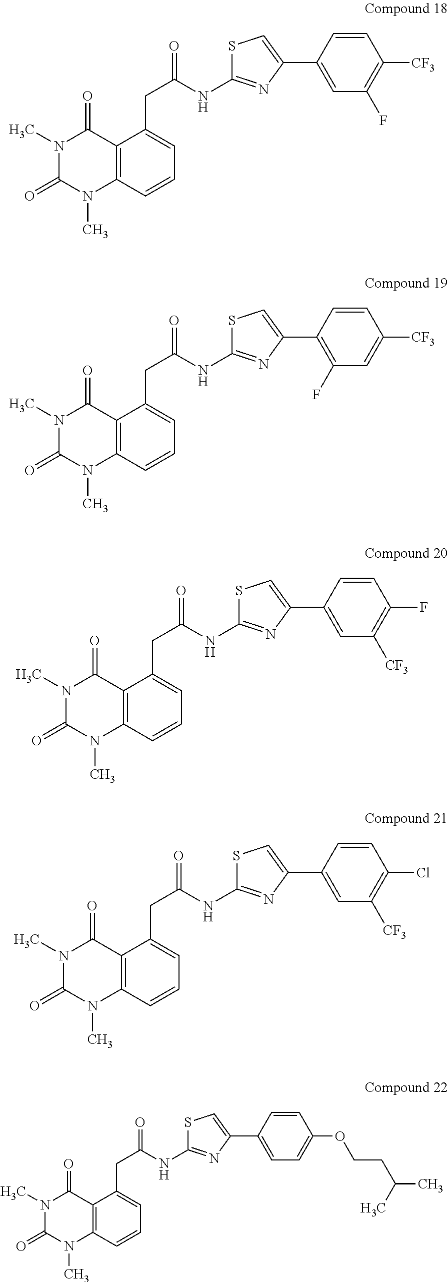 [SMILES: Cn1c(=O)c2c(CC(=O)Nc3nc(-c4ccc(C(F)(F)F)c(F)c4)cs3)cccc2n(C)c1=O ; Cn1c(=O)c2c(CC(=O)Nc3nc(-c4ccc(C(F)(F)F)cc4F)cs3)cccc2n(C)c1=O ; Cn1c(=O)c2c(CC(=O)Nc3nc(-c4ccc(F)c(C(F)(F)F)c4)cs3)cccc2n(C)c1=O ; Cn1c(=O)c2c(CC(=O)Nc3nc(-c4ccc(Cl)c(C(F)(F)F)c4)cs3)cccc2n(C)c1=O ; CC(C)CCOc1ccc(-c2csc(NC(=O)Cc3cccc4c3c(=O)n(C)c(=O)n4C)n2)cc1]

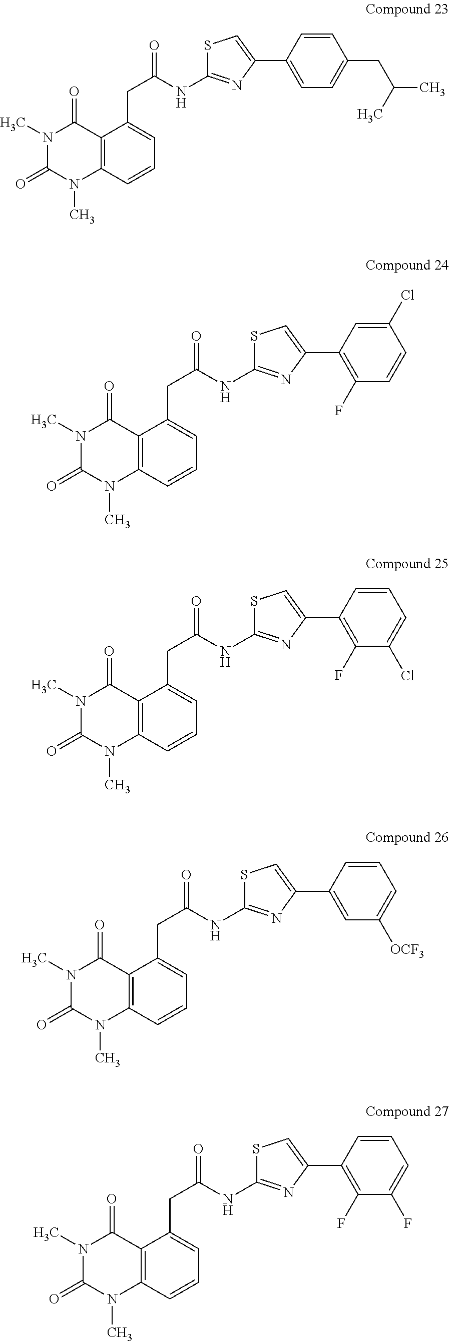 [SMILES: CC(C)Cc1ccc(-c2csc(NC(=O)Cc3cccc4c3c(=O)n(C)c(=O)n4C)n2)cc1 ; Cn1c(=O)c2c(CC(=O)Nc3nc(-c4cc(Cl)ccc4F)cs3)cccc2n(C)c1=O ; Cn1c(=O)c2c(CC(=O)Nc3nc(-c4cccc(Cl)c4F)cs3)cccc2n(C)c1=O ; Cn1c(=O)c2c(CC(=O)Nc3nc(-c4cccc(OC(F)(F)F)c4)cs3)cccc2n(C)c1=O ; Cn1c(=O)c2c(CC(=O)Nc3nc(-c4cccc(F)c4F)cs3)cccc2n(C)c1=O]

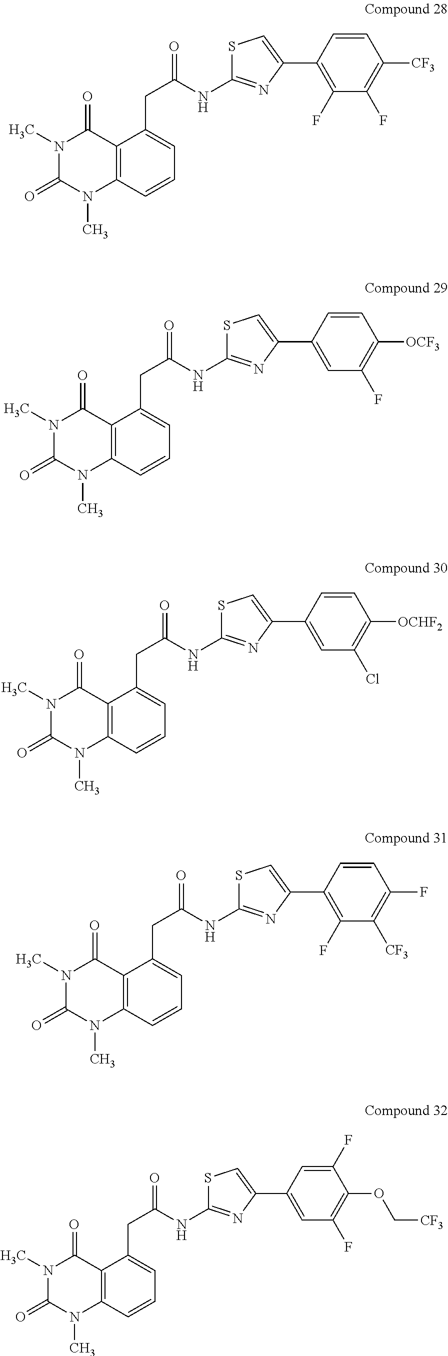 [SMILES: Cn1c(=O)c2c(CC(=O)Nc3nc(-c4ccc(C(F)(F)F)c(F)c4F)cs3)cccc2n(C)c1=O ; Cn1c(=O)c2c(CC(=O)Nc3nc(-c4ccc(OC(F)(F)F)c(F)c4)cs3)cccc2n(C)c1=O ; Cn1c(=O)c2c(CC(=O)Nc3nc(-c4ccc(OC(F)F)c(Cl)c4)cs3)cccc2n(C)c1=O ; Cn1c(=O)c2c(CC(=O)Nc3nc(-c4ccc(F)c(C(F)(F)F)c4F)cs3)cccc2n(C)c1=O ; Cn1c(=O)c2c(CC(=O)Nc3nc(-c4cc(F)c(OCC(F)(F)F)c(F)c4)cs3)cccc2n(C)c1=O]

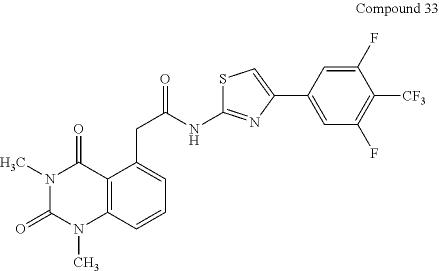 [SMILES: Cn1c(=O)c2c(CC(=O)Nc3nc(-c4cc(F)c(C(F)(F)F)c(F)c4)cs3)cccc2n(C)c1=O]

The preparation of above said compounds is described in WO2010004390. In one aspect, TRPA1 antagonists useful in the context of the invention are selected from those compounds generically or specifically disclosed in WO2010109287. Accordingly, TRPA1 antagonist useful in the context of the invention has the formula (III):

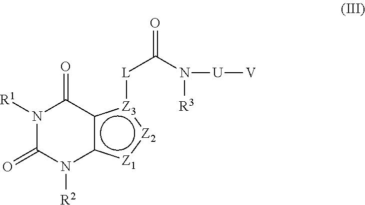

or a pharmaceutically acceptable salt thereof,
wherein,

Z₁ is NR^{a} or CR^{a};

Z₂ is NR^{b} or CR^{b};

Z₃ is N or C;

with the proviso that when Z₂ is CR^{b} then both Z₁ and Z₃ are not nitrogen at the same time;

at each occurrence, R^{a} and R^{b} which may be same or different, are independently selected from hydrogen, hydroxyl, cyano, halogen, substituted or unsubstituted alkyl, haloalkyl, alkenyl, alkynyl, cycloalkyl, cycloalkylalkyl, —(CR^{x}R^{u})ₙOR^{x}, —COR^{x}, —COOR^{x}, —CONR^{x}R^{y}, —S(O)ₙNR^{x}R^{y}, —NR^{x}R^{y}, —NR^{x}(CR^{x}R^{y})ₙOR^{x}, —(CH₂)ₙNR^{x}R^{y}, —(CH₂)ₙCHR^{x}R^{y}, —(CH₂)NR^{x}R^{y}, —NR^{x}(CR^{x}R^{y})ₙCONR^{x}R^{y}, —(CH₂)ₙNHCOR^{x}, —(CH₂)ₙNH(CH₂)ₙSO₂R^{x} and (CH₂)ₙNHSO₂R^{x};

alternatively either of R^{a} or R^{b} is absent;

R¹ and R², which may be same or different, are independently selected from hydrogen, hydroxyl, substituted or unsubstituted alkyl, haloalkyl, alkenyl, alkynyl, cycloalkyl, cycloalkylalkyl, arylalkyl, (CR^{x}R^{y})ₙOR^{x}, COR^{x}, COOR^{x}, CONR^{x}R^{y}, (CH₂)ₙNR^{x}R^{y}, (CH₂)ₙCHR^{x}R^{y}, (CH₂)NR^{x}R^{y} and (CH₂)ₙNHCOR^{x};

R³ is selected from hydrogen, substituted or unsubstituted alkyl, alkenyl, haloalkyl, alkynyl, cycloalkyl, cycloalkylalkyl, cycloalkenyl;

L is a linker selected from —(CR^{x}R^{y})ₙ—, —O—(CR^{x}R^{y})ₙ—, —C(O)—, —NR^{x}—, —S(O)ₘNR^{x}—, —NR^{x}(CR^{x}R^{y})ₙ— and —S(O)ₘNR^{x}(CR^{x}R^{y})ₙ;

U is selected from substituted or unsubstituted aryl, substituted or unsubstituted five membered heterocycles selected from the group consisting of thiazole, isothiazole, oxazole, isoxazole, thiadiazole, oxadiazole, pyrazole, imidazole, furan, thiophene, pyroles, 1,2,3-triazoles and 1,2,4-triazole; and substituted or unsubstituted six membered heterocycles selected from the group consisting of pyrimidine, pyridine and pyridazine;

V is selected from hydrogen, cyano, nitro, —NR^{x}R^{y}, halogen, hydroxyl, substituted or unsubstituted alkyl, alkenyl, alkynyl, cycloalkyl, cycloalkylalkyl, cycloalkenyl, haloalkyl, haloalkoxy, cycloalkylalkoxy, aryl, arylalkyl, biaryl, heteroaryl, heteroarylalkyl, heterocyclic ring and heterocyclylalkyl, —C(O)OR^{x}, —OR^{x}, —C(O)NR^{x}R^{y}, —C(O)R^{x} and —SO₂NR^{x}R^{y}; or U and V together may form an optionally substituted 3 to 7 membered saturated or unsaturated cyclic ring, that may optionally include one or more heteroatoms selected from O, S and N;

at each occurrence, R^{x} and R^{y} are independently selected from the group consisting of hydrogen, hydroxyl, halogen, substituted or unsubstituted alkyl, alkenyl, alkynyl, cycloalkyl, cycloalkylalkyl, cycloalkenyl, aryl, arylalkyl, heteroaryl, heteroarylalkyl, heterocyclic ring and heterocyclylalkyl; and
at each occurrence ‘m’ and ‘n’ are independently selected from 0 to 2, both inclusive.

Few representative TRPA1 antagonists useful in the context of the invention are mentioned below:

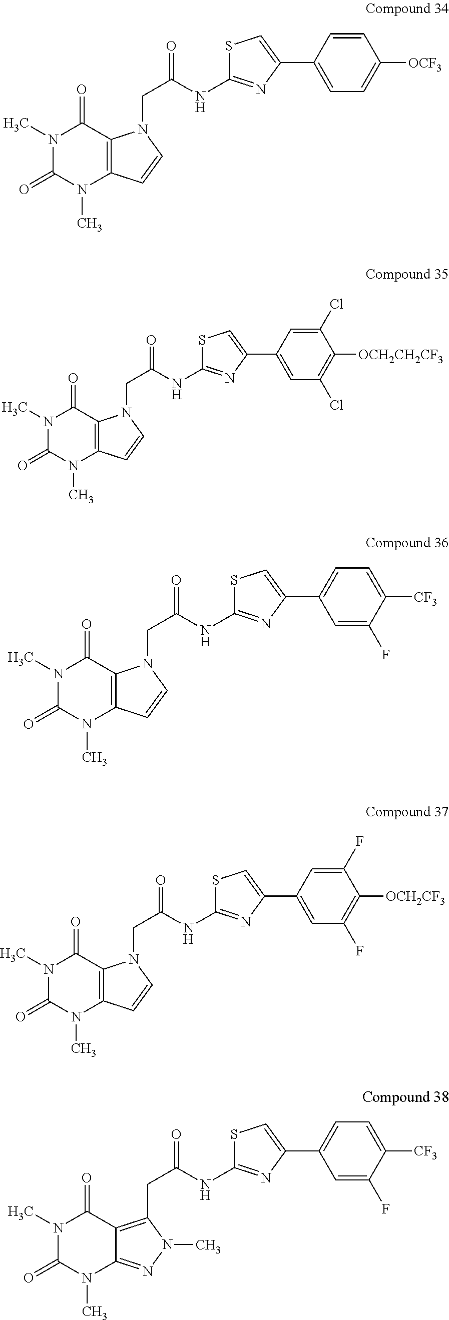

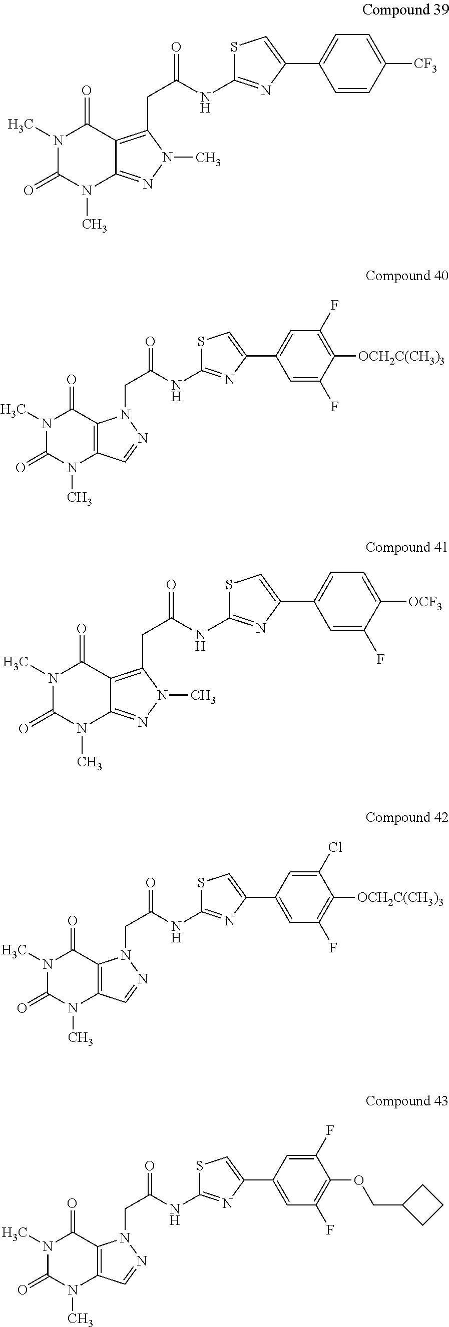

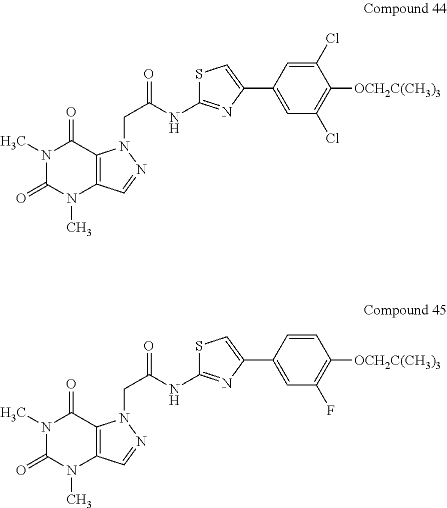

The preparation of above said compounds is described in WO2010109287.

In one aspect, TRPA1 antagonists useful in the context of the invention are selected from those compounds generically or specifically disclosed in WO 2010109334. Accordingly, TRPA1 antagonists useful in the context of the invention has the formula (IV)

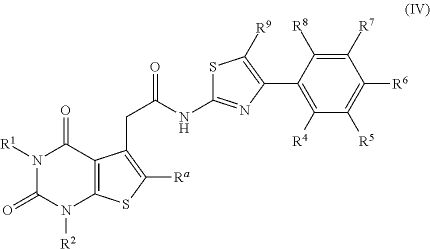

or a pharmaceutically-acceptable salt thereof.

wherein, R¹, R² and R^{a}, which may be the same or different, are each independently hydrogen or (C₁-C₄)alkyl;

R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl.

Few representative TRPA1 antagonists useful in the context of the invention are mentioned below:

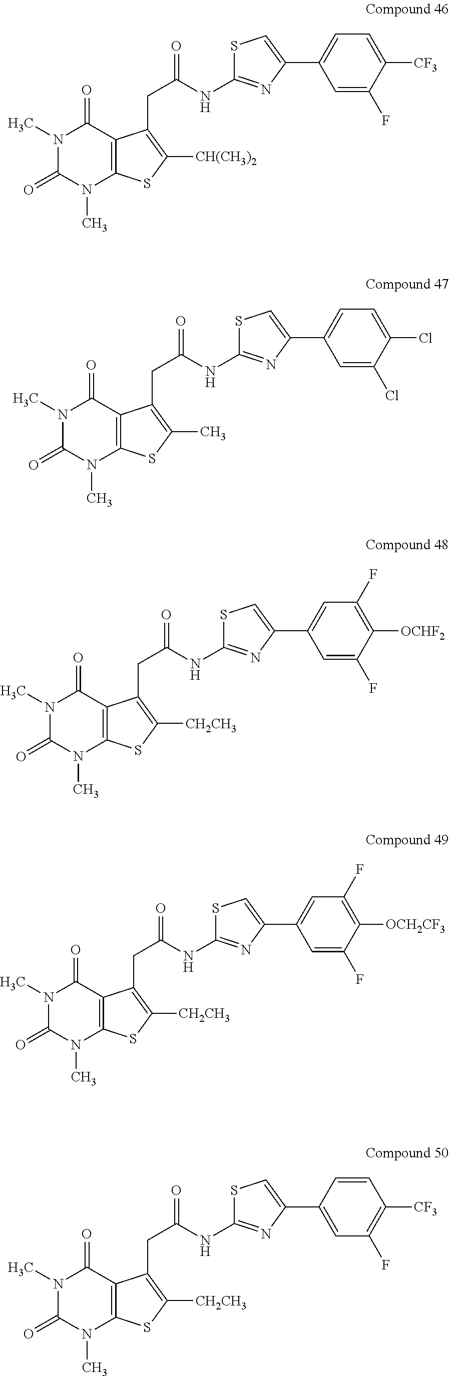

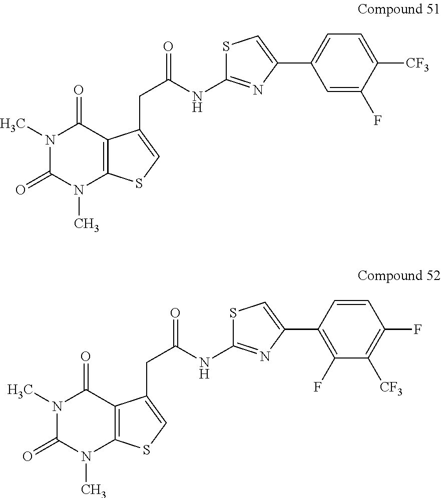 [SMILES: Cn1c(=O)c2c(CC(=O)Nc3nc(-c4ccc(C(F)(F)F)c(F)c4)cs3)csc2n(C)c1=O ; Cn1c(=O)c2c(CC(=O)Nc3nc(-c4ccc(F)c(C(F)(F)F)c4F)cs3)csc2n(C)c1=O]

The preparation of above said compounds is described in WO2010109334.

In one aspect, TRPA1 antagonists useful in the context of the invention are selected from those compounds generically or specifically disclosed in WO2010109329. Accordingly, TRPA1 antagonists useful in the context of the invention has the formula (V)

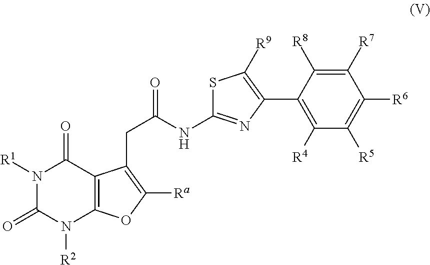

or a pharmaceutically acceptable salt thereof,
wherein, R¹, R² and R^{a} which may be the same or different, are each independently hydrogen or (C₁-C₄) alkyl; and

R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl.

Few representative TRPA1 antagonists useful in the context of the invention are mentioned below:

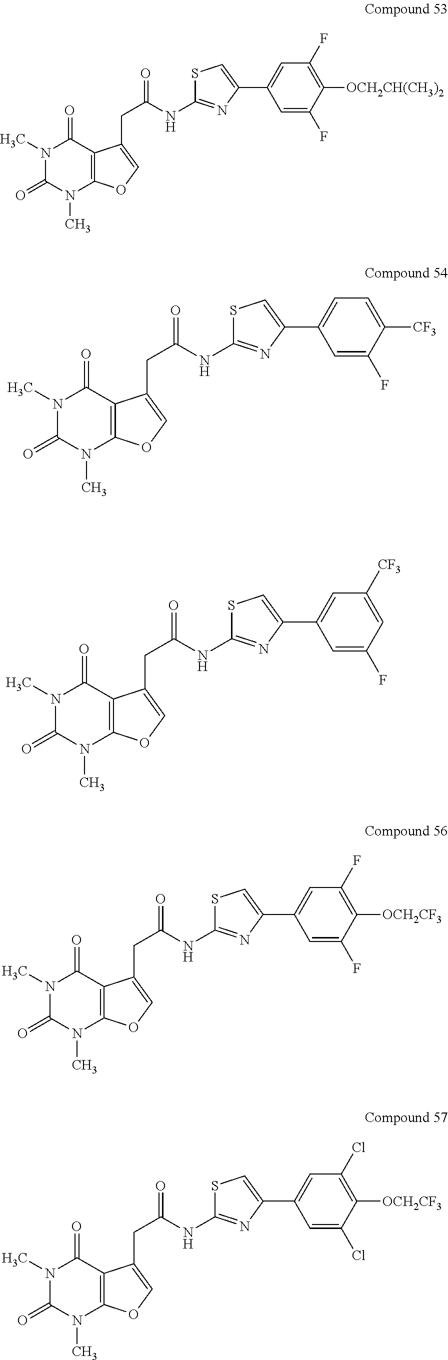

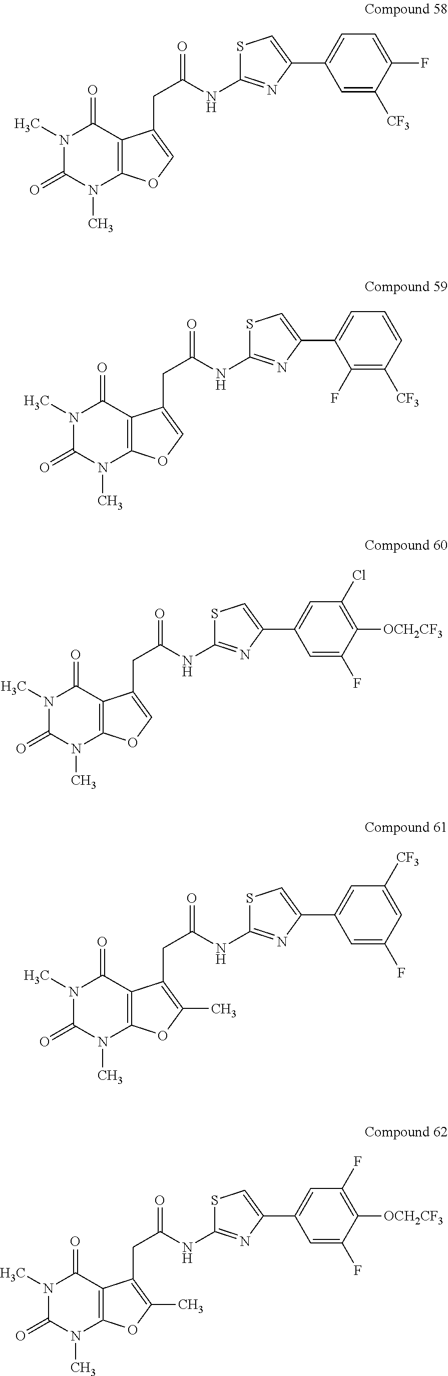

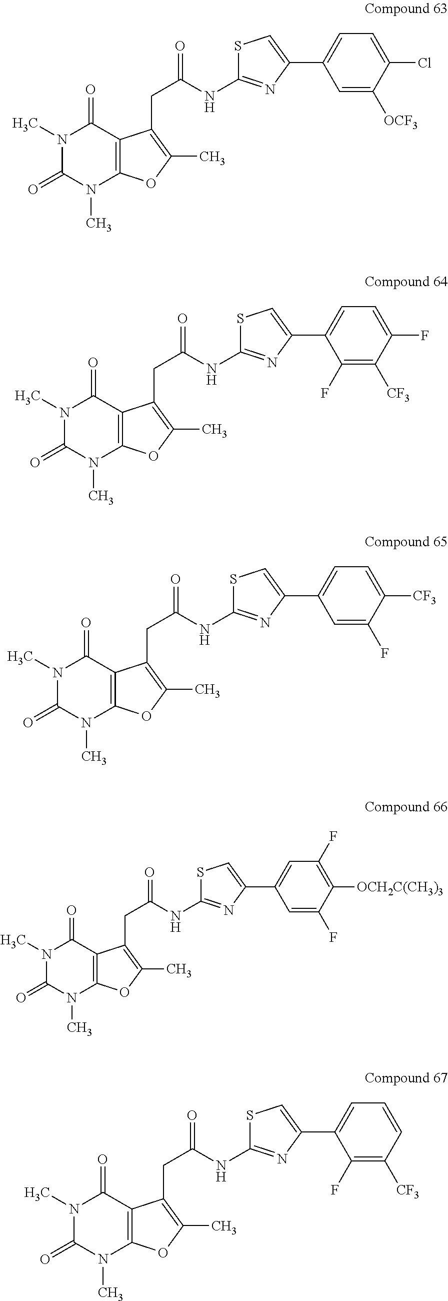

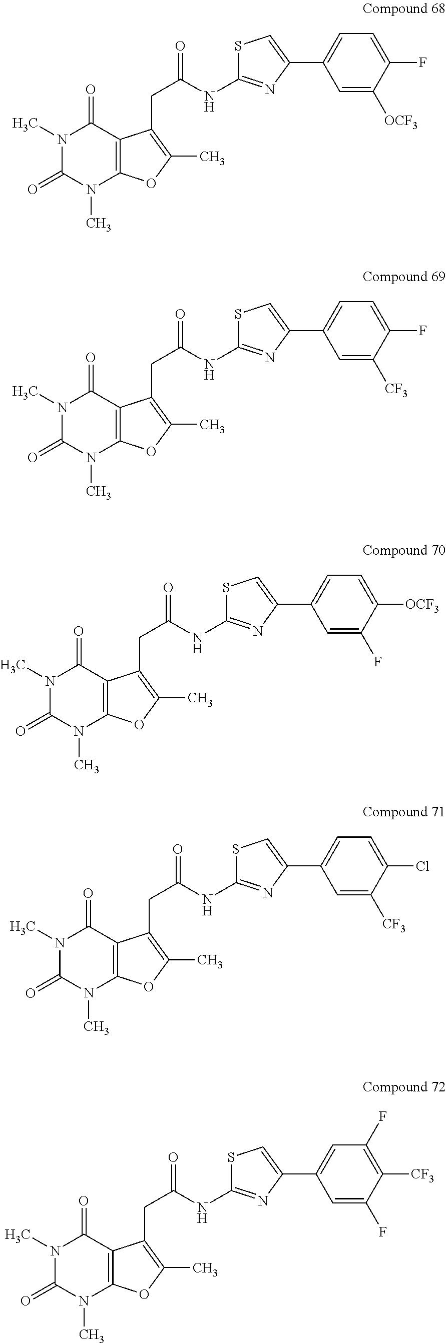 [SMILES: Cc1oc2c(c1CC(=O)Nc1nc(-c3ccc(F)c(OC(F)(F)F)c3)cs1)c(=O)n(C)c(=O)n2C ; Cc1oc2c(c1CC(=O)Nc1nc(-c3ccc(F)c(C(F)(F)F)c3)cs1)c(=O)n(C)c(=O)n2C ; Cc1oc2c(c1CC(=O)Nc1nc(-c3ccc(OC(F)(F)F)c(F)c3)cs1)c(=O)n(C)c(=O)n2C ; Cc1oc2c(c1CC(=O)Nc1nc(-c3ccc(Cl)c(C(F)(F)F)c3)cs1)c(=O)n(C)c(=O)n2C ; Cc1oc2c(c1CC(=O)Nc1nc(-c3cc(F)c(C(F)(F)F)c(F)c3)cs1)c(=O)n(C)c(=O)n2C]

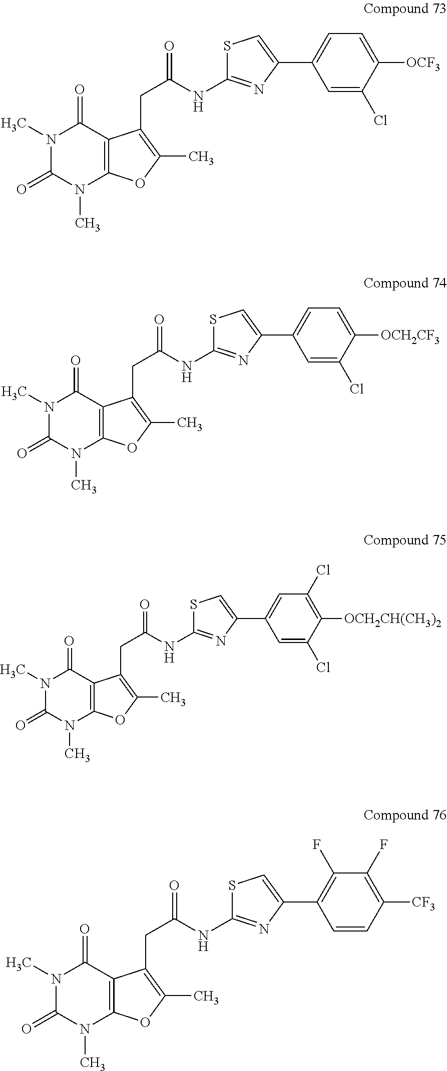

The preparation of above said compounds is described in WO2010109329.

In one aspect, TRPA1 antagonists useful in the context of the invention are selected from those compounds generically or specifically disclosed in WO2010109328. Accordingly, TRPA1 antagonists useful in the context of the invention has the formula (VI)

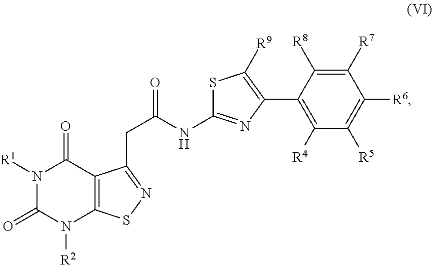

or a pharmaceutically-acceptable salt thereof.
wherein, R¹ and R², which may be the same or different, are each independently hydrogen or (C₁-C₄)alkyl; and

R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl.

Few representative TRPA1 antagonists useful in the context of the invention are mentioned below:

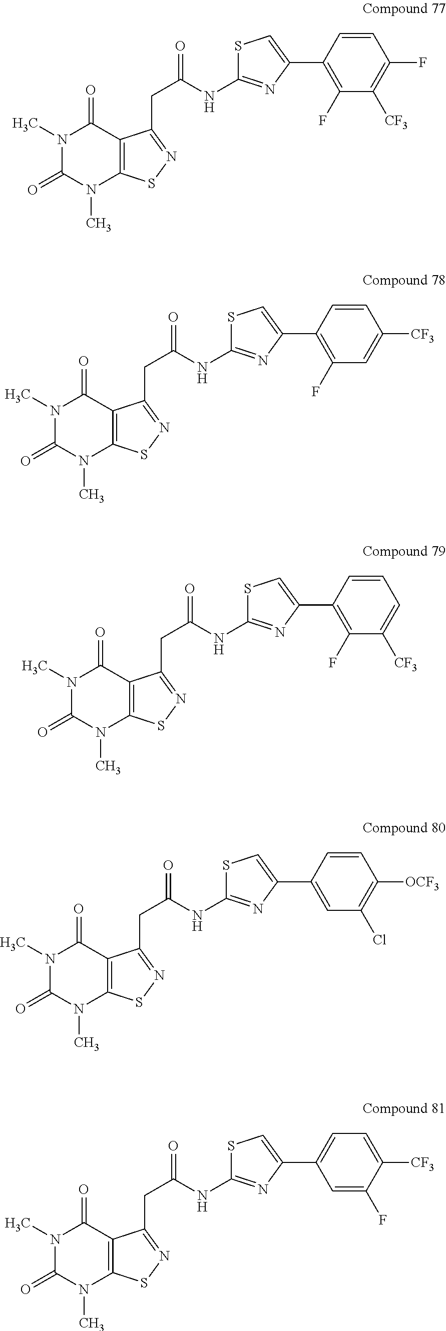 [SMILES: Cn1c(=O)c2c(CC(=O)Nc3nc(-c4ccc(F)c(C(F)(F)F)c4F)cs3)nsc2n(C)c1=O ; Cn1c(=O)c2c(CC(=O)Nc3nc(-c4ccc(C(F)(F)F)cc4F)cs3)nsc2n(C)c1=O ; Cn1c(=O)c2c(CC(=O)Nc3nc(-c4cccc(C(F)(F)F)c4F)cs3)nsc2n(C)c1=O ; Cn1c(=O)c2c(CC(=O)Nc3nc(-c4ccc(OC(F)(F)F)c(Cl)c4)cs3)nsc2n(C)c1=O ; Cn1c(=O)c2c(CC(=O)Nc3nc(-c4ccc(C(F)(F)F)c(F)c4)cs3)nsc2n(C)c1=O]

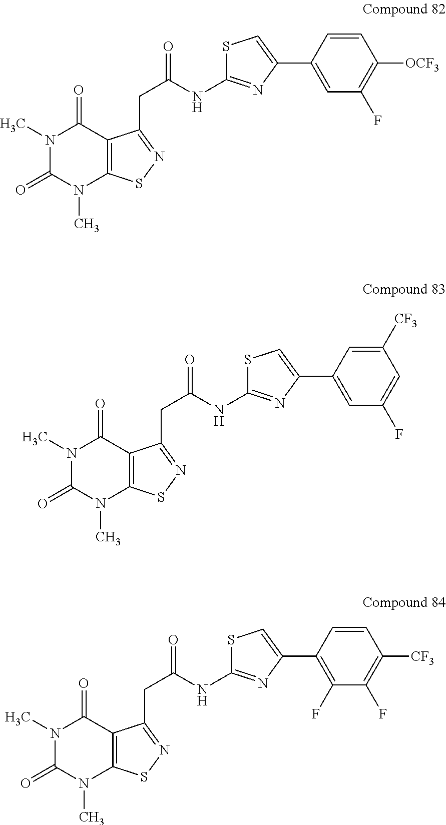 [SMILES: Cn1c(=O)c2c(CC(=O)Nc3nc(-c4ccc(OC(F)(F)F)c(F)c4)cs3)nsc2n(C)c1=O ; Cn1c(=O)c2c(CC(=O)Nc3nc(-c4cc(F)cc(C(F)(F)F)c4)cs3)nsc2n(C)c1=O ; Cn1c(=O)c2c(CC(=O)Nc3nc(-c4ccc(C(F)(F)F)c(F)c4F)cs3)nsc2n(C)c1=O]

The preparation of above said compounds is described in WO2010109328.

In one aspect, TRPA1 antagonists useful in the context of the invention are selected from those compounds generically or specifically disclosed in WO2010125469. Accordingly, TRPA1 antagonists useful in the context of the invention have the formulas (VIIa, VIIb and VIIc):

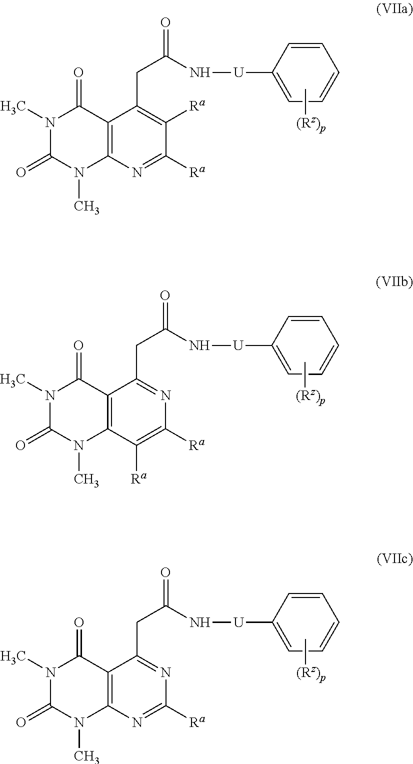

or pharmaceutically acceptable salt thereof,
wherein,

at each occurrence, R^{a} is selected from hydrogen, cyano, halogen, substituted or unsubstituted alkyl, haloalkyl, alkenyl, alkynyl, alkoxy, cycloalkyl and cycloalkylalkyl;

U is substituted or unsubstituted five membered heterocycle, for example selected from the group consisting of

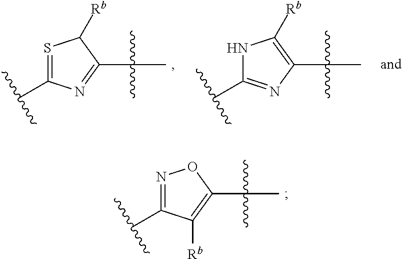

at each occurrence, R^{b} is independently selected from hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl;

at each occurrence, R^{z} is independently selected from halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring, heterocyclylalkyl, COOR^{x}, CONR^{x}R^{y}, S(O)ₘNR^{x}R^{y}, NR^{x}(CR^{x}R^{y})ₙOR^{x}, (CH₂)ₙNR^{x}R^{y}, NR^{x}(CR^{x}R^{y})ₙCONR^{x}R^{y}, (CH₂)ₙNHCOR^{x}, (CH₂)ₙNH(CH₂)ₙSO₂R^{x} and (CH₂)ₙNHSO₂R^{x};

at each occurrence, R^{x} and R^{y} are independently selected from hydrogen, hydroxyl, halogen, substituted or unsubstituted alkyl, alkenyl, alkynyl, cycloalkyl, cycloalkylalkyl, cycloalkenyl, aryl, arylalkyl, heteroaryl, heteroarylalkyl, heterocyclic ring and heterocyclylalkyl;

at each occurrence, ‘m’ and ‘n’ are independently selected from 0 to 2, both inclusive; and ‘p’ is independently selected from 0 to 5, both inclusive.

Few representative TRPA1 antagonists useful in the context of the invention are mentioned below:

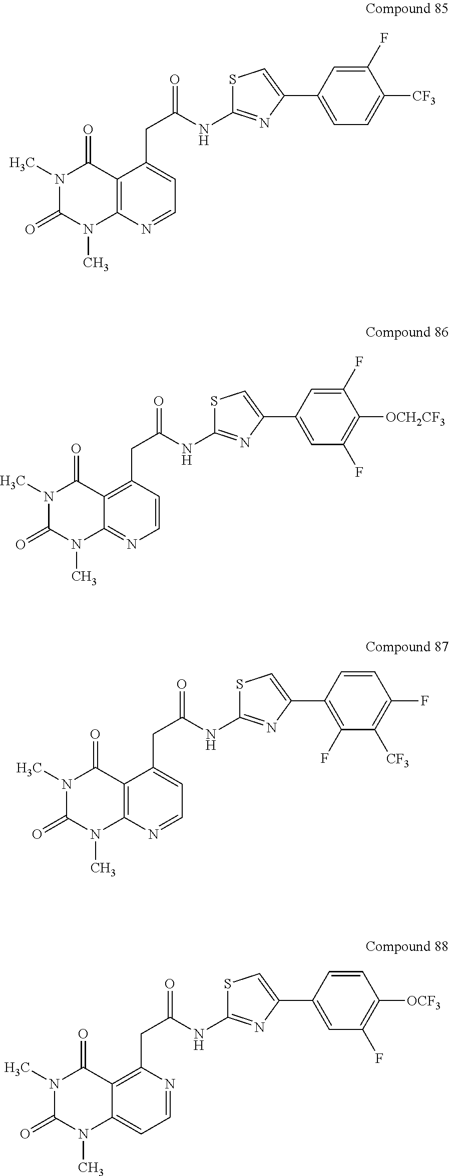

The preparation of above said compounds is described in WO2010125469.

In one aspect, the TRPA1 antagonist useful in the context of the invention is Compound 89:

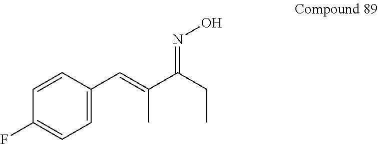

In one embodiment, the TRPA1 antagonist useful in the context of the invention is Compound 90:

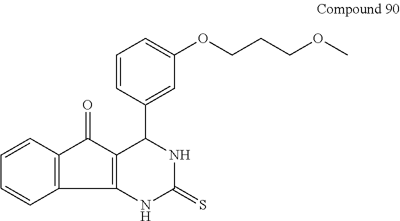 [SMILES: COCCCOc1cccc(C2NC(=S)NC3=C2C(=O)c2ccccc23)c1]

In an embodiment, TRPA1 antagonists useful in the context of the invention has the formula

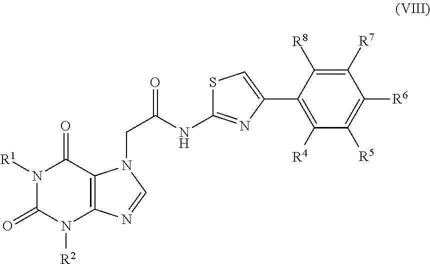

or a pharmaceutically-acceptable salt thereof
wherein,
R¹, R² and R^{a}, which may be the same or different, are each independently hydrogen or (C₁-C₄)alkyl;
R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl.

A representative TRPA1 antagonist useful in the context of the invention is Compound 91:

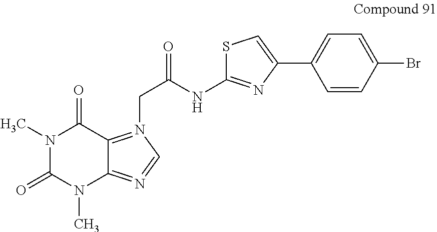

The Compound 91 may be prepared, for example, by following the process provided for the preparation of similar compounds in PCT publication No. WO2007073505.

In another aspect, TRPA1 antagonists useful in the context of the invention are selected from those compounds generically or specifically disclosed in WO2011114184. Accordingly, a TRPA1 antagonist useful in the context of the invention has the formula (IX):

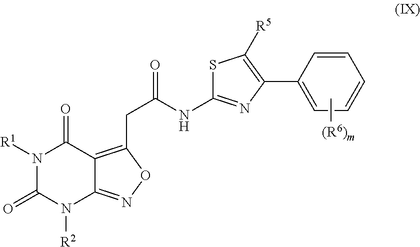

or a pharmaceutically-acceptable salt thereof

wherein at each occurrence, R¹ and R² are independently selected from hydrogen or substituted or unsubstituted alkyl;

at each occurrence, R⁵ is selected from hydrogen, halogen or substituted or unsubstituted alkyl;

at each occurrence, R⁶ is selected from hydrogen, cyano, nitro, halogen, hydroxyl, substituted or unsubstituted alkyl, alkenyl, alkynyl, cycloalkyl, cycloalkylalkyl, cycloalkenyl, haloalkyl, haloalkoxy, cycloalkylalkoxy, aryl, arylalkyl, biaryl, heteroaryl, heteroarylalkyl, heterocyclic ring and heterocyclylalkyl.

A representative TRPA1 antagonist useful in the methods of the invention is mentioned below:

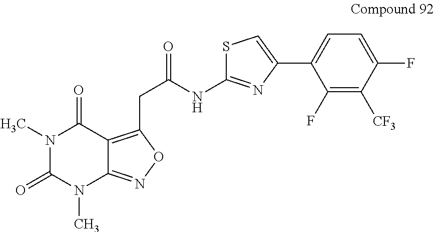 [SMILES: Cn1c(=O)c2c(CC(=O)Nc3nc(-c4ccc(F)c(C(F)(F)F)c4F)cs3)onc2n(C)c1=O]

The preparation of above said compounds is described in WO2011114184.

In another aspect, TRPA1 antagonist useful in the context of the invention has the formula (X):

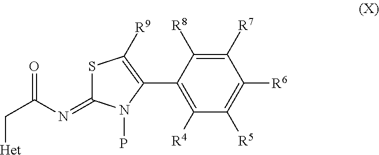

wherein, ‘Het’ is selected from groups consisting of

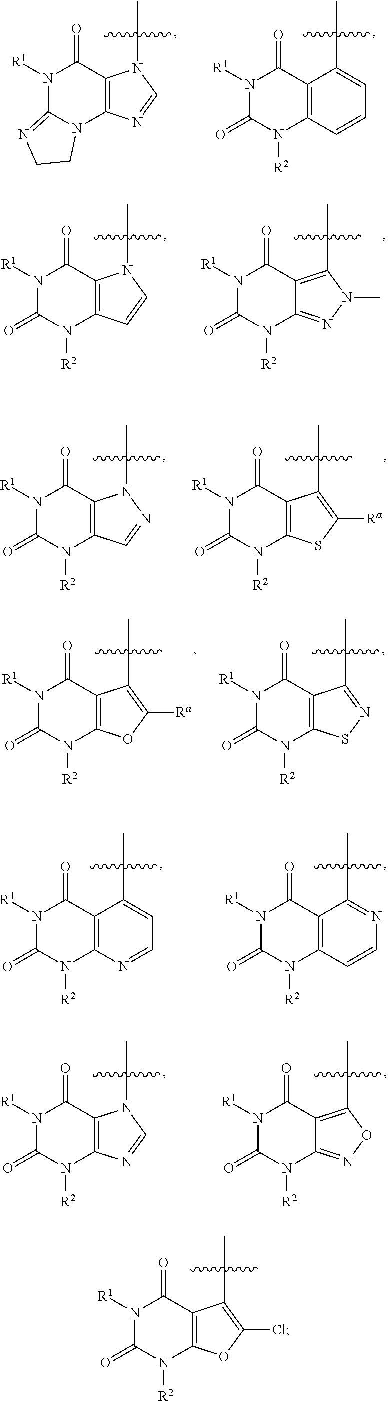

P is selected from

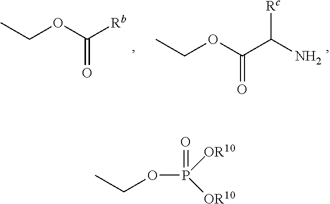

R¹, R² and R^{a}, which may be the same or different, are each independently hydrogen or (C₁-C₄) alkyl;

R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl;

R^{b} and R^{c} independently selected from hydrogen, substituted or unsubstituted alkyl arylalkyl, amino acid and heterocyclic ring;

R¹⁰ is selected from hydrogen, alkyl, arylalkyl and pharmaceutically acceptable cation.

Few representative TRPA1 antagonists useful in the context of the invention are mentioned below:

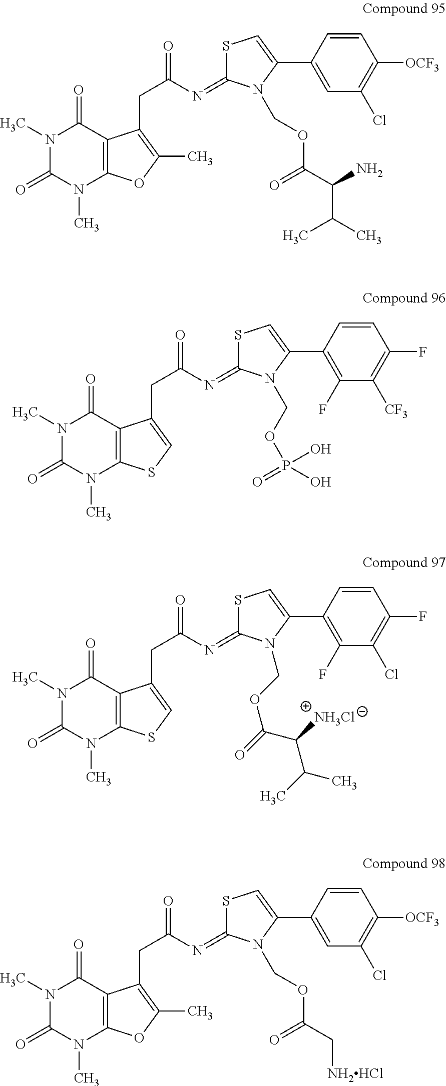

In another aspect, TRPA1 antagonists useful in the context of the invention are selected from those compounds generically or specifically disclosed in WO2011114184. Accordingly, TRPA1 antagonist useful in the context of the invention has the formula (XI):

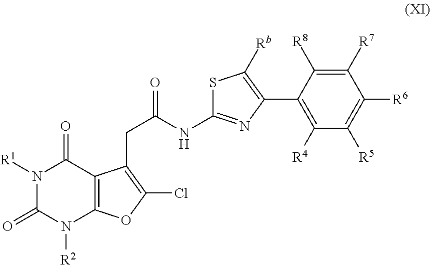

or a pharmaceutically acceptable salt thereof,

wherein, R¹, and R² are independently hydrogen or (C₁-C₄)alkyl; and

R⁴, R⁵, R⁶, R⁷, R⁸ and R⁹, which may be same or different, are each independently selected from halogen haloalkyl, dialkylamino, and haloalkoxy.

Few representative TRPA1 antagonists useful in the context of the invention are mentioned below:

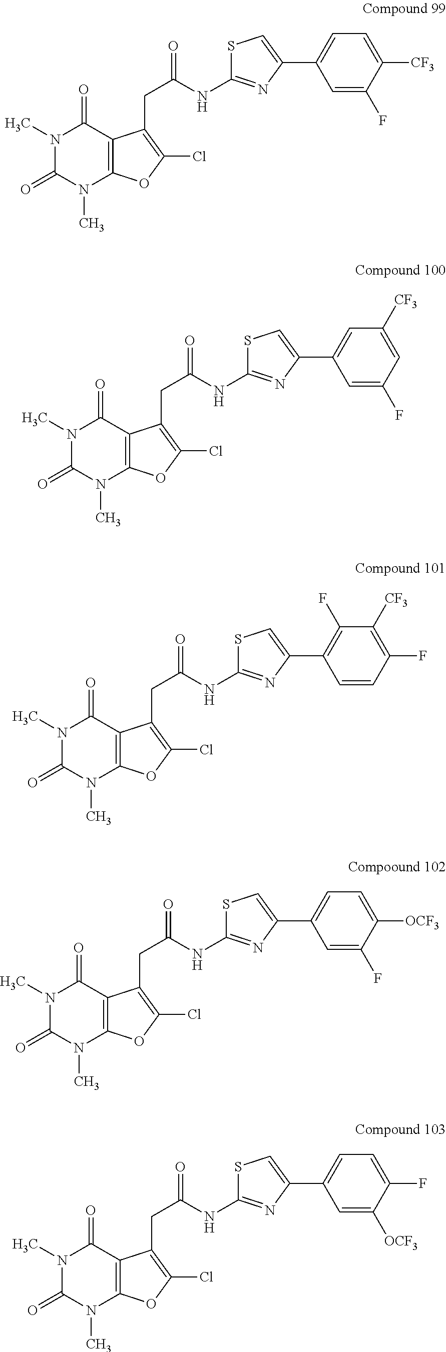 [SMILES: Cn1c(=O)c2c(CC(=O)Nc3nc(-c4ccc(C(F)(F)F)c(F)c4)cs3)c(Cl)oc2n(C)c1=O ; Cn1c(=O)c2c(CC(=O)Nc3nc(-c4cc(F)cc(C(F)(F)F)c4)cs3)c(Cl)oc2n(C)c1=O ; Cn1c(=O)c2c(CC(=O)Nc3nc(-c4ccc(F)c(C(F)(F)F)c4F)cs3)c(Cl)oc2n(C)c1=O ; Cn1c(=O)c2c(CC(=O)Nc3nc(-c4ccc(OC(F)(F)F)c(F)c4)cs3)c(Cl)oc2n(C)c1=O ; Cn1c(=O)c2c(CC(=O)Nc3nc(-c4ccc(F)c(OC(F)(F)F)c4)cs3)c(Cl)oc2n(C)c1=O]

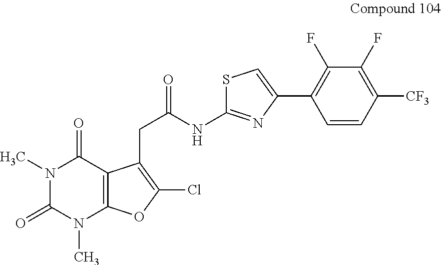 [SMILES: Cn1c(=O)c2c(CC(=O)Nc3nc(-c4ccc(C(F)(F)F)c(F)c4F)cs3)c(Cl)oc2n(C)c1=O]

The preparation of above said compounds is described in WO2011114184.

In an aspect, TRPA1 antagonists useful in the context of the invention, is selected from one of the following formulae: (XII) or (D)

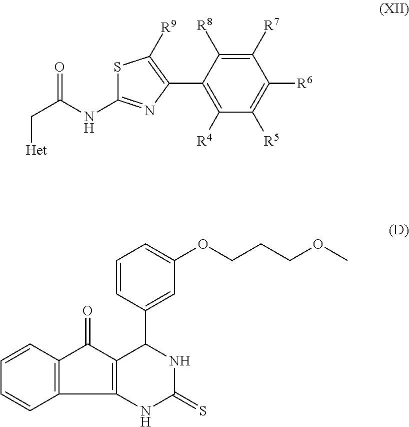

or a pharmaceutically-acceptable salt thereof, wherein, ‘Het’ is selected from the group consisting of

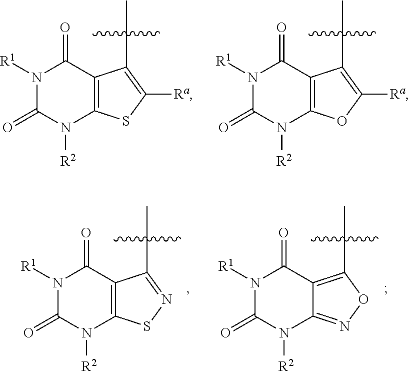

R¹, R² and R^{a}, which may be the same or different, are each independently hydrogen or (C₁-C₄) alkyl;
R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl.

Few representative TRPA1 antagonists of the formula (XII) useful in the context of the invention are compound 52, compound 73 and compound 84 as described above.

The anticholinergic agent, as contemplated herein, includes tiotropium, oxitropium, ipratropium, glycopyrrolate and aclidinium or salts thereof. The salt may be present in the form of their isomers, polymorphs, and solvates, including hydrates, all of which are included in the scope of the invention.

In another embodiment, the present invention relates to a pharmaceutical composition comprising synergistically effective amount of a TRPA1 antagonist having an IC₅₀ for inhibiting human TRPA1 receptor activity of less than 1 micromolar having structure of formulae: (XII) or (D)

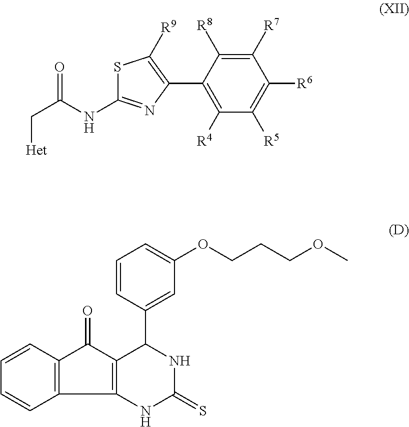

or a pharmaceutically-acceptable salt thereof, wherein, ‘Het’ is selected from the group consisting of

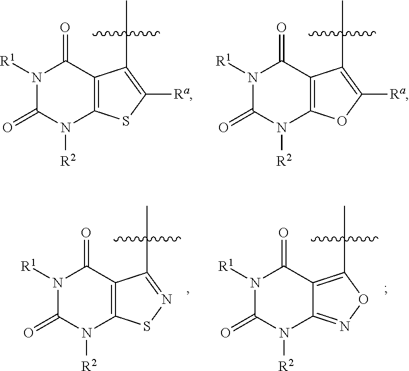

R¹, R² and R^{a}, which may be the same or different, are each independently hydrogen or (C₁-C₄) alkyl;
R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl and an anticholinergic agent. Preferably, the TRPA1 antagonist of the present invention has an IC₅₀ for inhibiting human TRPA1 receptor activity of less than 500 nanomolar, or more preferably less than 250 nanomolar, as measured by a method described herein.

In yet another embodiment, the present invention relates to a pharmaceutical composition comprising synergistically effective amount of a TRPA1 antagonist having structure of formula: and an anticholinergic agent.

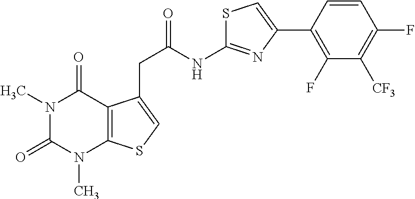 [SMILES: Cn1c(=O)c2c(CC(=O)Nc3nc(-c4ccc(F)c(C(F)(F)F)c4F)cs3)csc2n(C)c1=O]

In another embodiment, there is provided a pharmaceutical composition comprising synergistically effective amount of a TRPA1 antagonist as contemplated herein and an anticholinergic agent in a weight ratio ranging from about 1:0.0001 to about 1:10000.

In an embodiment, the present invention relates to a pharmaceutical composition comprising synergistically effective amount of a TRPA1 antagonist having structure of formula:

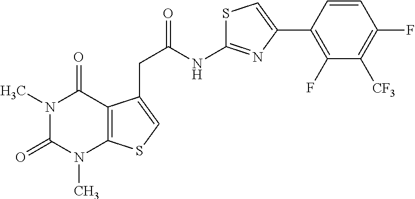 [SMILES: Cn1c(=O)c2c(CC(=O)Nc3nc(-c4ccc(F)c(C(F)(F)F)c4F)cs3)csc2n(C)c1=O]

and an anticholinergic agent selected from a group consisting of tiotropium, oxitropium, ipratropium, glycopyrrolate and aclidinium or salts thereof. In first aspect of this embodiment, the anticholinergic agent is tiotropium. In second aspect of this embodiment, the anticholinergic agent is ipratropium. In third aspect of this embodiment, the anticholinergic agent is aclidinium. In another aspect of this embodiment, the pharmaceutical composition is a fixed dose combination.

The pharmaceutical composition of the present invention may be administered orally, nasally, intra-tracheally, parenterally, transdermally, transmucosal, inhalation or by any other route that a physician or a health-care provider may determine to be appropriate. Preferably, the route of administration is oral or by inhalation.

In yet another aspect of this embodiment, the composition is for inhalation administration and the TRPA1 antagonist and the anticholinergic agent are present in a weight ratio from about 1:0.001 to about 1:300.

As contemplated herein, the active ingredients may be administered together in a single dosage form or they may be administered in different dosage forms. They may be administered at the same time or they may be administered either close in time or remotely, such as, where one drug is administered in the morning and the second drug is administered in the evening. The combination may be used prophylactically or after the onset of symptoms has occurred.

In a preferred embodiment, both the active ingredients i.e., TRPA1 antagonist and the anticholinergic agent are formulated as a pharmaceutical composition suitable for administration by the same route (e.g., both the actives by oral or inhalation route), or by different routes (e.g., one active by oral and the other active by inhalation route).

The pharmaceutical compositions for oral administration may be in conventional forms, for example, tablets, capsules, granules (synonymously, “beads” or “particles” or “pellets”), suspensions, emulsions, powders, dry syrups, and the like. The capsules may contain granule/pellet/particle/mini-tablets/mini-capsules containing the active ingredients. The amount of the active agent that may be incorporated in the pharmaceutical composition may range from about 1% w/w to about 98% w/w or from about 5% w/w to about 90% w/w.

The pharmaceutical compositions for parenteral administration include but are not limited to solutions/suspension/emulsion for intravenous, subcutaneous or intramuscular injection/infusion, and implants. The pharmaceutical compositions for transdermal or transmucosal administration include but are not limited to patches, gels, creams, ointments and the like.

As set forth above, the pharmaceutical composition includes at least one pharmaceutically acceptable excipient, which includes but is not limited to one or more of the following; diluents, glidants and lubricants, preservatives, buffering agents, chelating agents, polymers, gelling agents/viscosifying agents, surfactants, solvents and the like.

In an embodiment, the present invention provides a process for the preparing a pharmaceutical composition comprising TRPA1 antagonist and an anticholinergic agent and a pharmaceutically acceptable excipient, wherein the composition is in the form of a fixed dose combination formulation. The process comprises admixing TRPA1 antagonist with the anticholinergic agent. Alternately, the process comprises formulating TRPA1 antagonist and the anticholinergic agent in such a way that they are not in intimate contact with each other.

In another embodiment, the invention relates to a process for preparing a pharmaceutical composition comprising TRPA1 antagonist, an anticholinergic agent and a pharmaceutically acceptable excipient, wherein the composition is in the form of kit comprising separate formulations of TRPA1 antagonist and the anticholinergic agent.

The process for making the pharmaceutical composition may for example include, (1) granulating either or both the active ingredients, combined or separately, along with pharmaceutically acceptable carriers so as to obtain granulate, and (2) converting the granulate into suitable dosage forms for oral administration. The typical processes involved in the preparation of the pharmaceutical combinations include various unit operations such as mixing, sifting, solubilizing, dispersing, granulating, lubricating, compressing, coating, and the like. These processes, as contemplated by a person skilled in the formulation art, have been incorporated herein for preparing the pharmaceutical composition of the present invention.

### Methods of Treatment

Asthma and COPD are major chronic diseases related to airway obstruction. The Global Initiative for Chronic Obstructive Lung Disease provides guidelines for the distinction between asthma and COPD. Asthma is believed to be a chronic inflammatory disease wherein the airflow limitation is more or less reversible while it is more or less irreversible in case of COPD. Asthma among other things is believed to be triggered by inhalation of sensitizing agents (like allergens) unlike noxious agents (like particles and certain gases) in case of COPD. Though both are believed to have an inflammatory component, the inflammation in asthma is believed to be mostly eosinophilic and CD-4 driven, while it is believed to be mostly neutrophilic and CD-8 driven in COPD.

Asthma is clinically classified according to the frequency of symptoms, forced expiratory volume in 1 second (FEV₁), peak expiratory flow rate and severity (e.g., acute, intermittent, mild persistent, moderate persistent, and severe persistent) Asthma may also be classified as allergic (extrinsic) or non-allergic (intrinsic), based on whether symptoms are precipitated by allergens or not. Asthma can also be categorized according to following types viz., nocturnal asthma, bronchial asthma, exercise induced asthma, occupational asthma, seasonal asthma, silent asthma, and cough variant asthma.

COPD, also known as chronic obstructive lung disease (COLD), chronic obstructive airway disease (COAD), or chronic obstructive respiratory disease (CORD), is believed to be the co-occurrence of chronic bronchitis (characterized by a long-term cough with mucus) and emphysema (characterized by destruction of the lungs over time), a pair of commonly co-existing diseases of the lungs in which the airways become narrowed. This leads to a limitation of the flow of air to and from the lungs, causing shortness of breath. An acute exacerbation of COPD is a sudden worsening of COPD symptoms (shortness of breath, quantity and color of phlegm) that typically lasts for several days and is believed to be triggered by an infection with bacteria or viruses or by environmental pollutants. Based on the FEV₁ values, COPD can be classified as mild, moderate, severe and very severe.

It is believed that reduction of eosinophil or neutrophil count and increase in FEV1 are important components of the treatment of respiratory disorders such as asthma and COPD. It is also believed that there exits an inverse correlation between eosinophil or neutrophil count and FEV1 value in human. For example, Ulrik CS, 1995 (Peripheral eosinophil counts as a marker of disease activity in intrinsic and extrinsic asthma; Clinical and Experimental Allergy; 1995, Volume 25, pages 820-827) discloses the relationship between eosinophil count and severity of asthmatic symptoms. It describes that in childhood and adulthood subjects, there exists an inverse correlation between number of eosinophils and FEV1% (r=−0.75, P<0.001, and r=−0.80, P<0.001, respectively).

Further, Peleman RA, 1999 (The cellular composition of induced sputum in chronic obstructive pulmonary disease; European Respiratory Journal; 1999, Volume 13, pages 839-843) discloses the relationship between percentage of neutrophils and FEV1 in patients with COPD. It describes that in patients with COPD, an inverse correlation was noted between percentage of neutrophils and FEV1 (r=−0.48, p<0.05).

Various classes of drugs are currently being used for the treatment and/or prophylaxis of respiratory disorders like asthma and COPD. Some of the classes of such drugs are leukotriene receptor antagonists, antihistamines, beta-2 agonists, anticholinergic agents and corticosteroids.

Human airways are innervated by a generous supply of efferent, cholinergic, parasympathetic autonomic nerves. Motor nerves derived from the vagus form ganglia within and around the walls of the airways. Release of acetylcholine (ACh) at these sites results in stimulation of muscarinic receptors and subsequent airway smooth muscle contraction and release of secretions from the submucosal airway glands. Epithelial and inflammatory cells also generate ACh and express functional muscarinic receptors. Recent findings indicate that ACh, acting on muscarinic receptors, may contribute to the pathophysiology and pathogenesis of asthma and COPD.

Anticholinergic agents are believed to reverse the action of vagally derived acetylcholine on airway smooth muscle contraction. Vagal tone is increased in airway inflammation associated with asthma and COPD; this results from exaggerated acetylcholine release and enhanced expression of downstream signaling components in airway smooth muscle. Vagally derived acetylcholine also regulates mucus production in the airways. Anticholinergic drugs can effectively inhibit accelerated decline of lung function. Further, anticholinergic agents can achieve reductions in airway remodeling and lung function decline in addition to its effects as a bronchodilator (Reinoud et. al. “Review: Muscarinic receptor signaling in the pathophysiology of asthma and COPD”, Respiratory Research 2006, 7:73).

Thus, it is believed that though the therapeutic outcomes of these two classes of drugs, the TRPA1 antagonists and the anticholinergic agent are similar to some extent, the mechanism of actions may vary to a good extent and thus the therapeutic effect of their combination in the treatment of respiratory disorders is highly unpredictable. Particularly, the therapeutic effect of the combination of TRPA1 antagonist and an anticholinergic agent is highly unpredictable.

The inventors of the present invention have surprisingly found that a pharmaceutical composition comprising TRPA1 antagonist and an anticholinergic agent are more effective in the treatment of respiratory disorders, and provide better therapeutic value when compared to both the actives alone (when administered individually) for the treatment of respiratory disorders.

In an embodiment, the present invention relates to a method of treating a respiratory disorder in a subject, said method comprising administering to the subject the pharmaceutical composition comprising synergistically effective amount of a TRPA1 antagonist having an IC₅₀ for inhibiting human TRPA1 receptor activity of less than 1 micromolar and an anticholinergic agent. In an aspect of this embodiment, the TRPA1 antagonist has an IC₅₀ for inhibiting human TRPA1 receptor activity of less than 1 micromolar having structure of formulae: (XII) or (D)

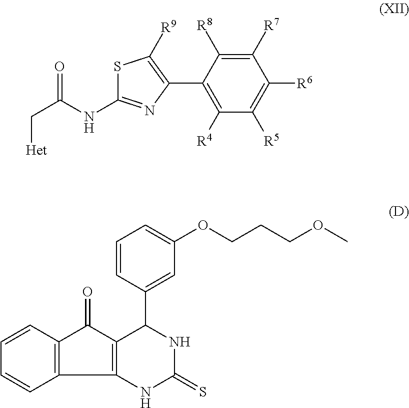

or a pharmaceutically-acceptable salt thereof, wherein, ‘Het’ is selected from the group consisting of

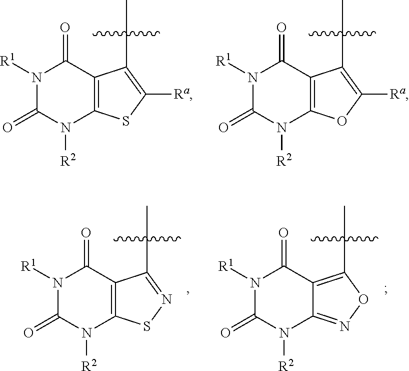

R¹, R² and R^{a}, which may be the same or different, are each independently hydrogen or (C₁-C₄) alkyl;
R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl.

In a further embodiment, the present invention relates to a method of treating a respiratory disorder in a subject, said method comprising administering the subject a pharmaceutical composition comprising synergistically effective amount of a TRPA1 antagonist having an IC₅₀ for inhibiting human TRPA1 receptor activity of less than 1 micromolar and an anticholinergic agent selected from a group consisting of tiotropium, oxitropium, ipratropium, glycopyrrolate and aclidinium or salts thereof.

In a further embodiment, the present invention relates to use of synergistically effective amount of a TRPA1 antagonist having an IC₅₀ for inhibiting human TRPA1 receptor activity of less than 1 micromolar and an anticholinergic agent in the preparation of a pharmaceutical composition of the present invention for the treatment of a respiratory disorder in a subject. In an aspect of this embodiment, the TRPA1 antagonist has an IC₅₀ for inhibiting human TRPA1 receptor activity of less than 1 micromolar having structure of formulae: (XII) or (D)

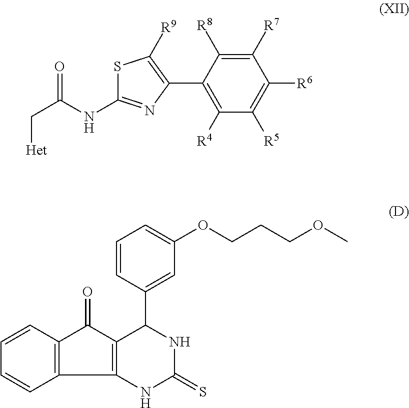

or a pharmaceutically-acceptable salt thereof, wherein, ‘Het’ is selected from the group consisting of

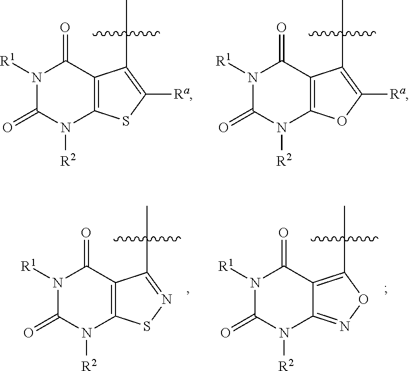

R¹, R² and R^{a}, which may be the same or different, are each independently hydrogen or (C₁-C₄) alkyl;
R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl.

In a further embodiment, the present invention relates to a pharmaceutical composition comprising synergistically effective amount of a TRPA1 antagonist having an IC₅₀ for inhibiting human TRPA1 receptor activity of less than 1 micromolar and an anticholinergic agent for the treatment of a respiratory disorder in a subject.

In an embodiment, the present invention relates to a method of treating a respiratory disorder in a subject, said method comprising administering to the subject the pharmaceutical composition comprising synergistically effective amount of a TRPA1 antagonist having structure of formula:

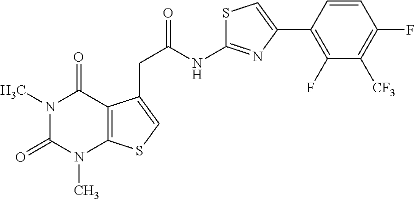 [SMILES: Cn1c(=O)c2c(CC(=O)Nc3nc(-c4ccc(F)c(C(F)(F)F)c4F)cs3)csc2n(C)c1=O]

and an anticholinergic agent. In an aspect of this embodiment, the anticholinergic agent is selected from a group consisting of tiotropium, oxitropium, ipratropium, glycopyrrolate and aclidinium or salts thereof.

In an embodiment, the present invention relates to a method of treating COPD by reducing neutrophil count in a subject, said method comprising administering to the subject the pharmaceutical composition comprising synergistically effective amount of a TRPA1 antagonist having structure of formula:

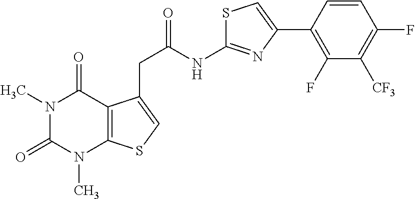 [SMILES: Cn1c(=O)c2c(CC(=O)Nc3nc(-c4ccc(F)c(C(F)(F)F)c4F)cs3)csc2n(C)c1=O]

and an anticholinergic agent. In an aspect of this embodiment, the anticholinergic agent selected from a group consisting of tiotropium, oxitropium, ipratropium, glycopyrrolate and aclidinium or salts thereof. In another aspect of this embodiment, the respiratory disorder is asthma. In another aspect of the embodiment, the composition is for inhalation administration.

In an embodiment, the present invention relates to a method of reducing neutrophil count in a subject, said method comprising administering to the subject the pharmaceutical composition comprising synergistically effective amount of a TRPA1 antagonist having structure of formula:

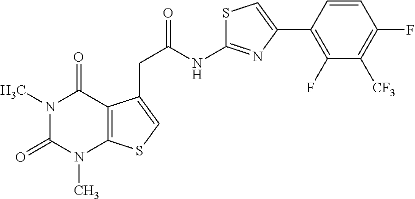 [SMILES: Cn1c(=O)c2c(CC(=O)Nc3nc(-c4ccc(F)c(C(F)(F)F)c4F)cs3)csc2n(C)c1=O]

and an anticholinergic agent. In an aspect of this embodiment, the anticholinergic agent selected from a group consisting of tiotropium, oxitropium, ipratropium, glycopyrrolate and aclidinium or salts thereof. In another aspect of the embodiment, the composition is for inhalation administration.

In an embodiment, the present invention relates to a method of treating asthma by inhibiting airway resistance in a subject, said method comprising administering to the subject the pharmaceutical composition comprising synergistically effective amount of a TRPA1 antagonist having structure of formula:

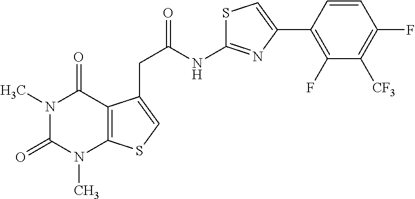 [SMILES: Cn1c(=O)c2c(CC(=O)Nc3nc(-c4ccc(F)c(C(F)(F)F)c4F)cs3)csc2n(C)c1=O]

and an anticholinergic agent. In an aspect of this embodiment, the anticholinergic agent selected from a group consisting of tiotropium, oxitropium, ipratropium, glycopyrrolate and aclidinium or salts thereof. In another aspect of this embodiment, the respiratory disorder is asthma. In another aspect of the embodiment, the composition is for inhalation administration.

In an embodiment, the present invention relates to a method of inhibiting airway resistance in a subject, said method comprising administering to the subject the pharmaceutical composition comprising synergistically effective amount of a TRPA1 antagonist having structure of formula:

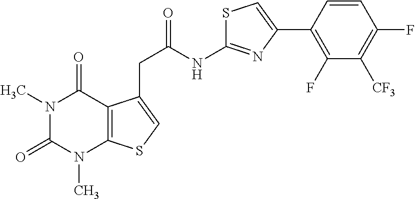 [SMILES: Cn1c(=O)c2c(CC(=O)Nc3nc(-c4ccc(F)c(C(F)(F)F)c4F)cs3)csc2n(C)c1=O]

and an anticholinergic agent. In an aspect of this embodiment, the anticholinergic agent selected from a group consisting of tiotropium, oxitropium, ipratropium, glycopyrrolate and aclidinium or salts thereof. In another aspect of the embodiment, the composition is for inhalation administration.

In another embodiment, the present invention relates to use of synergistically effective amount of a TRPA1 antagonist having structure of formula:

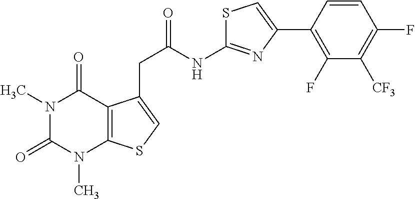 [SMILES: Cn1c(=O)c2c(CC(=O)Nc3nc(-c4ccc(F)c(C(F)(F)F)c4F)cs3)csc2n(C)c1=O]

and an anticholinergic agent in the preparation of a pharmaceutical composition of the present invention for the treatment of a respiratory disorder in a subject. In an aspect of this embodiment, the anticholinergic agent is selected from a group consisting of tiotropium, oxitropium, ipratropium, glycopyrrolate and aclidinium or salts thereof.

In a further embodiment, the present invention relates to a pharmaceutical composition comprising synergistically effective amount of a TRPA1 antagonist having structure of formula:

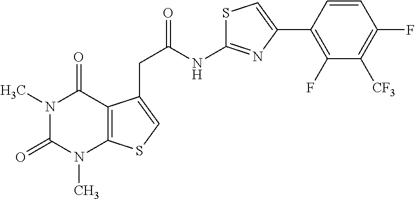 [SMILES: Cn1c(=O)c2c(CC(=O)Nc3nc(-c4ccc(F)c(C(F)(F)F)c4F)cs3)csc2n(C)c1=O]

and an anticholinergic agent for the treatment of a respiratory disorder in a subject.

The therapeutically effective amount of TRPA1 antagonist to be administered per day ranges from about 10 mcg/kg to about 20 mg/kg, and preferably from about 50 mcg/kg to about 15 mg/kg.

In one embodiment of the present invention the therapeutically effective amount of tiotropium to be administered per day ranges from about 5 mcg to about 50 mcg and preferably from about 10 mcg to about 36 mcg. Preferably, the discrete dosage strengths of tiotropium or its salt are 15 mcg or 18 mcg or 20 mcg or 22 mcg or 22.5 mcg or 25 mcg or 36 mcg.

In one embodiment of the present invention the therapeutically effective amount of ipratropium to be administered per day ranges from about 10 mcg to about 200 mcg and preferably from about 20 mcg to about 150 mcg. Preferably the discrete dosage strengths of ipratropium or its salt are 34 mcg or 42 mcg or 68 mcg or 84 mcg or 102 mcg or 126 mcg or 146 mcg or 168 mcg.

In one embodiment of the present invention the therapeutically effective amount of aclidinium bromide to be administered per day ranges from 150 mcg to about 800 mcg, and preferably from about 200 mcg to about 600 mcg. Preferably the discrete dosage strengths of aclidinium or its salt are 200 mcg or 400 mcg or 600 mcg or 800 mcg.

The optimal dose of the active ingredient or the combination of the active ingredients can vary as a function of the severity of disease, route of administration, composition type, the patient body weight, the age and the general state of mind of the patient, and the response to behavior to the active ingredient or the combination of the active ingredients.

In the pharmaceutical composition as described herein, the active ingredient may be in the form of a single dosage form (i.e., fixed-dose formulation in which both the active ingredients are present together) or they may be divided doses, formulated separately, each in its individual dosage forms but as part of the same therapeutic treatment, program or regimen, either once daily or two/three/four times a day.

Alternately, the invention relates to a pharmaceutical composition wherein the composition is in the form of kit comprising separate formulations of TRPA1 antagonist and the anticholinergic agent. The separate formulations are to be administered by same or different routes, either separately, simultaneously, or sequentially, where the sequential administration is close in time or remote in time. For sequential administration, the period of time may be in the range from 10 min to 12 hours.

Various animal models have been used for the evaluation of the therapeutic efficacy of drug candidates for respiratory disorders like asthma and COPD. For example, commonly used strategy for evaluation of drug candidates in asthma is the allergen sensitization and challenge method. The commonly used such model is the ovalbumin (OVA) sensitization and challenge in laboratory animals. Another model that can be used is the methacholine challenge test by using invasive whole body plethysmograph.

A commonly used model for evaluation of drug candidates in COPD involves the chronic exposure of the animal to SO₂ or tobacco/cigarette smoke. The model is believed to generate sloughing of epithelial cells, increase in the mucus secretions, increase in the polymorphonuclear cells and pulmonary resistance, and increase in the airway hyper-responsiveness (in rats).

Another model that can be used for evaluation of drug candidates in COPD involves the exposure of animals (e.g., rats) to lipopolysaccharide (LPS). The exposure to LPS is believed to result in the influx of neutrophils in the lungs, a condition that is believed to be one of the characteristics of COPD.

It will be understood that various modifications may be made to the embodiments disclosed herein. Therefore the above description should not be construed as limiting, but merely as exemplifications of preferred embodiments. Other arrangements and methods may be implemented by those skilled in the art without departing from the scope and spirit of this invention.

The following examples are provided to enable one skilled in the art to practice the invention and are merely illustrative of the invention. The examples should not be read as limiting the scope of the invention.

### EXAMPLES

### Example 1

### Determination of IC₅₀ Value of TRPA1 Antagonists

The human IC₅₀ values were measured by the following method:

The inhibition of TRPA1 receptor activation was measured as inhibition of allylisothiocyanate (AITC) induced cellular uptake of radioactive calcium.

Test compound solution was prepared in a suitable solvent.

Human TRPA1 expressing CHO cells were grown in suitable medium. Cells were treated with test compounds followed by addition of AITC.

Cells were washed, lysed and the radioactivity in the lysate was measured in Packard Top count after addition of liquid scintillant.

The concentration response curves for compounds were plotted as a % of maximal response obtained in the absence of test antagonist, and the IC₅₀ values were calculated from such concentration response curve by nonlinear regression analysis using GraphPad PRISM software.

TABLE 1
TRPA1 antagonists having a human IC₅₀ for inhibiting human
TRPA1 receptor activity of less than 1 micromolar.

| Compound No | hTRPA1 IC₅₀ values | |
|---|---|---|
| 1 | 920.9 | nM |
| 2 | 381.8 | nM |
| 3 | 73.35 | nM |
| 4 | 98.32 | nM |
| 5 | 66.28 | nM |
| 6 | 97.42 | nM |
| 7 | 47.37 | nM |
| 8 | 55.02 | nM |
| 9 | 102.5 | nM |
| 10 | 46.74 | nM |
| 11 | 46.27 | nM |
| 12 | 51.68 | nM |
| 13 | 48.21 | nM |
| 14 | 60.42 | nM |
| 15 | 53.57 | nM |
| 16 | 58.94 | nM |
| 17 | 56.02 | nM |
| 18 | 13.38 | nM |
| 19 | 26.13 | nM |
| 20 | 20.09 | nM |
| 21 | 48.18 | nM |
| 22 | 79.77 | nM |
| 23 | 43.93 | nM |
| 24 | 138.1 | nM |
| 25 | 58.55 | nM |
| 26 | 47.91 | nM |
| 27 | 65.45 | nM |
| 28 | 6.49 | nM |
| 29 | 11.38 | nM |
| 30 | 34.03 | nM |
| 31 | 17.3 | nM |
| 32 | 5.96 | nM |
| 33 | 5.37 | nM |
| 34 | 38.46 | nM |
| 35 | 18.05 | nM |
| 36 | 49.92 | nM |
| 37 | 12.26 | nM |
| 38 | 15.92 | nM |
| 39 | 26.56 | nM |
| 40 | 22.82 | nM |
| 41 | 11.04 | nM |
| 42 | 11.38 | nM |
| 43 | 18.37 | nM |
| 44 | 8.36 | nM |
| 45 | 26.39 | nM |
| 46 | 41.31 | nM |
| 47 | 33.61 | nM |
| 48 | 18.12 | nM |
| 49 | 3.98 | nM |
| 50 | 16.73 | nM |
| 51 | 4.84 | nM |
| 52 | 2.49 | nM |
| 53 | 18.20 | nM |
| 54 | 17.74 | nM |
| 55 | 2.15 | nM |
| 56 | 3.38 | nM |
| 57 | 1.45 | nM |
| 58 | 11.88 | nM |
| 59 | 2.21 | nM |
| 60 | 3.54 | nM |
| 61 | 2.93 | nM |
| 62 | 1.68 | nM |
| 63 | 9.04 | nM |
| 64 | 4.52 | nM |
| 65 | 6.65 | nM |
| 66 | 3.63 | nM |
| 67 | 13.59 | nM |
| 68 | 4.84 | nM |
| 69 | 7.10 | nM |
| 70 | 12.57 | nM |
| 71 | 3.18 | nM |
| 72 | 4.16 | nM |
| 73 | 8.54 | nM |
| 74 | 5.29 | nM |
| 75 | 3.34 | nM |
| 76 | 4.02 | nM |
| 77 | 5.60 | nM |
| 78 | 10.57 | nM |
| 79 | 5.29 | nM |
| 80 | 6.28 | nM |
| 81 | 6.74 | nM |
| 82 | 8.04 | nM |
| 83 | 4.40 | nM |
| 84 | 5.35 | nM |
| 85 | 8.92 | nM |
| 86 | 6.91 | nM |
| 87 | 19.32 | nM |
| 88 | 11.45 | nM |
| 89 | 98.44 | nM |
| 90 | 5.61 | nM |
| 91 | 451.4 | nM |
| 92 | 17.08 | nM |
| 95 | 88.50 | nM |
| 96 | 559.3 | nM |
| 97 | 21.91 | nM |
| 98 | 54.29 | nM |
| 99 | 5.06 | nM |
| 100 | 5.15 | nM |
| 101 | 10.10 | nM |
| 102 | 7.67 | nM |
| 103 | 27.41 | nM |
| 104 | 7.58 | nM |

### Example 2

### Animal Studies for the Combination of Compound 52 and Tiotropium Bromide

The effect of Compound 52, tiotropium bromide and their combination on methacholine challenge test in male Dunkin Hartley guinea pig was evaluated using invasive whole body plethysmograph (Elan™ R C, Buxco apparatus). Animals and grouped as described in Table 2.

TABLE 2

| | | Dose | | | |
|---|---|---|---|---|---|
| | | Tiotropium | Com- | | Number of |
| | | bromide | pound 52 | | animals |
| Group | Treatment | (mcg/kg) | (mg/kg) | Route | (N) |
| 1 | Vehicle | — | — | — | 9 |
| | control | | | | |
| 2 | Tiotropium | 0.5 | — | i.v* | 7 |
| | bromide | | | | |
| 3 | Com- | — | 10 | i.p** | 5 |
| | pound 52 | | | | |
| 4 | Combination | 0.5 | 10 | i.v*/i.p** | 6 |

*intravenous;
**intraperitonial

Animals were anesthetized by intraperitonial injection of urethane (1 g/kg). The surgical process carried out in the aseptic area. Right jugular vein of anesthetized animal was exposed and cannulated with fine bore polythene tube. Trachea was also cannulated for artificial respiration and to measure airway resistance. The prepared animal was fixed in the invasive whole body plethysmograph with artificial respiration (tidal volume −7 ml). Methacholine challenge test was performed and the recordings were taken by the following schedule.
First log period: Baseline −5 min
Second log period: Normal saline −3 min
Third log period: Methacholine −3 min

This protocol was performed for all groups. Compound 52 (10 mg/kg, i.p.) was injected 4 hour prior to methacholine challenge. Tiotropium (0.5 mcg/kg, i.v) was given at second log period. In third log period, the animal was injected with methacholine (60 mcg/kg/2 ml, i.v). The methacholine induced bronchoconstriction (expressed as airway resistance (RI value in cmH2O*sec/ml)) was recorded by Buxco apparatus.

TABLE 3

| | | Mean airway | % inhibition in |
|---|---|---|---|
| | | resistance | airway resistance |
| Group | Treatment | (Mean ± SEM) | (Mean ± SEM) |
| 1 | Vehicle | 1.36 ± 0.17 | — |
| | control | | |
| 2 | Tiotropium | 1.51 ± 0.32 | −10.7 ± 23.4 |
| | bromide | | |
| 3 | Compound 52 | 0.77 ± 0.11 | 37.9 ± 6.7 |
| 4 | Combination | 0.38 ± 0.05 | 72.2 ± 3.8*^{#} |

*p < 0.05-treated groups vs Group 1;
^{#}p < 0.01 vs Group 2

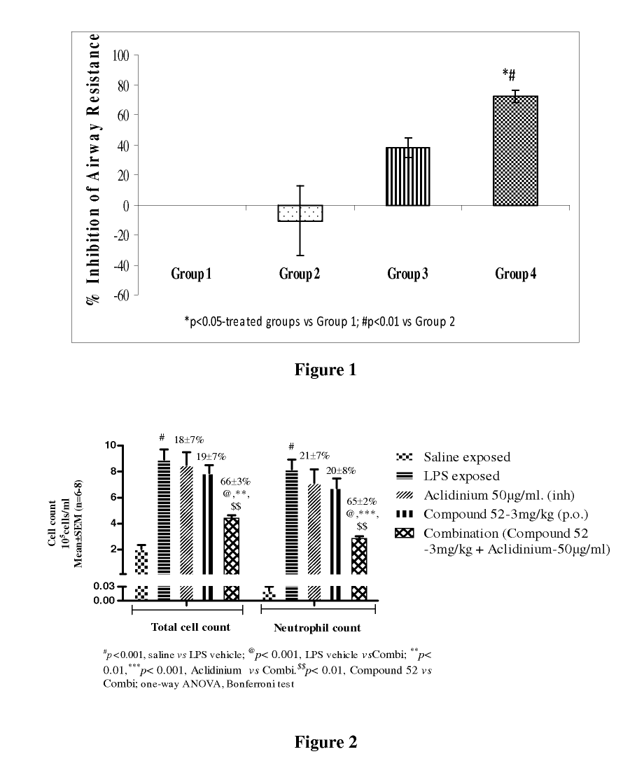

Combination of Compound 52 and tiotropium bromide was found to produce significantly superior inhibition (synergistic effect) in airway resistance compared to the individual and sum of the activity of both in methacholine challenge as shown in Table 3 and FIG. 1.

### Example 3

### Animal Studies for the Combination of Compound 52 and Aclidinium Bromide

The effect of combination of Compound 52 and aclidinium bromide on LPS induced neutrophilia in male SD rats was evaluated. Animals were grouped as mentioned in Table 4.

TABLE 4

| | | Dose | | |
|---|---|---|---|---|
| | | Aclidinium | Compound 52 | Exposure |
| Groups | Treatment (n) | (mcg/ml, inh) | (mg/kg, p.o.) | to |
| 1 | Saline control (6) | — | — | saline |
| 2 | LPS control (8) | — | — | LPS |
| 3 | Compound 52 (8) | — | 3 | |
| 4 | Aclidinium | 50 | — | |
| | bromide (8) | | | |
| 5 | Combination (8) | 50 | 3 | |

*All groups were exposed to LPS (0111: B4) (100 mcg/ml) for 40 min except vehicle control group

LPS was nebulized at concentration of 100 mcg ml⁻¹ for 40 min at 0.4 ml/min and a pressure of 1.7 psig in a Perspex exposure chamber (1.5×1×1 ft) fitted with nebulizer (RCI Hudson). Control animals were given saline exposure under similar conditions. All the animals were treated with compounds as mentioned in Table 4. Compound 52 (3 mg/kg/5 ml) was administered orally 2 h prior to LPS and aclidinium (50 mcg/ml) was nebulized at the rate of 0.05 ml/min and exposed to the animals for 3 min prior LPS exposure.

Bronchoalveolar lavage (BAL) was done after 4 h of LPS exposure. Animals were anesthetized with urethane (1.2 gm/kg/5 mL, i.p. in normal saline) and BAL was done with PBS (3 ml). This procedure was repeated three times and the BAL fluid was pooled for the measurement of total leukocyte. Remaining BAL fluid was centrifuged immediately at 4000 rpm for 20 min. The pellet formed in the bottom of the tube was used for the smear preparation for differential leukocyte count estimation. The smeared slides were fixed by alcohol and stained using Leishman's stain further carried for differential leukocyte count.

TABLE 5

| | | Dose | | No of neutrophils |
|---|---|---|---|---|
| | | Aclidinium | Compound 52 | (% inhibition; |
| Groups | Treatment (n) | (mcg/ml, inh) | (mg/kg, p.o.) | mean ± S.E.M.) |
| 1 | Saline (6) | — | — | 0.02 ± 0.01 |
| 2 | LPS (8) | — | — | 8.16 ± 0.76^{#} |
| 3 | Compound 52 (8) | — | 3 | 6.73 ± 0.72 (20.4 ± 7.7) |
| 4 | Aclidinium | 50 | — | 7.13 ± 1.03 (21.1 ± 7.2) |
| | bromide (8) | | | |
| 5 | Combination (8) | 50 | 3 | 2.84 ± 0.19 (65.4 ± 2.3)^{@,}***^{,$$} |

^{#}p < 0.001, saline vs LPS vehicle;
^{@}p < 0.001, LPS vehicle vs Combination;
***p < 0.001, Aclidinium vs Combination;
^{$$}p < 0.01, Compound 52 vs Combination; one-way ANOVA, Bonferroni test.

Combination of compound 52 with aclidinium showed significant inhibition in LPS induced neutrophilia compared to the individual treatments (Table 5 and FIG. 2). Compound 52 in combination with aclidinium showed significant synergy in inhibition of neutrophilia in LPS model in SD rats.

### Example 4

### Animal Studies for the Combination of Compound 52 and Tiotropium

The effect of combination of Compound 52 and tiotropium on LPS induced neutrophilia in female SD rats was evaluated. Female SD rats were grouped as mentioned in Table 6. Compound 52 (3 mg/kg/5 ml) and tiotropium (1 mcg/ml as inhalation for 3 min at the rate of 0.05 ml/min) were administered 2 and 1 hour prior to LPS exposure respectively. LPS was nebulized at concentration of 100 mcg ml⁻¹ for 40 min at 0.4 ml min⁻¹ and a pressure of 1.7 psig in a perspex exposure chamber (1.5×1×1 ft) fitted with nebulizer (RCI Hudson). Control animals were given saline exposure under similar conditions.

Bronchoalveolar lavage (BAL) was done after 4 h of LPS exposure. Animal was anesthetized with urethane (1.2 gm/kg/5 mL, i.p. in normal saline) and BAL was done with PBS (3 ml). This procedure was repeated three times and the BAL fluid was pooled for the measurement of total leukocyte. BAL fluid was centrifuged immediately at 4000 rpm for 20 min and the pellet formed in the bottom of the tube was used for the smear preparation for differential leukocyte count estimation.

TABLE 6

| | | Dose | | |
|---|---|---|---|---|
| | | Tiotropium | Compound 52 | Exposure |
| Groups | Treatment (n) | (mcg/ml, inh) | (mg/kg, p.o.) | to |
| 1 | Saline (6) | — | — | saline |
| 2 | LPS (8) | — | — | LPS |
| 3 | Compound 52 (8) | — | 3 | |
| 4 | Tiotropium (8) | 1 | — | |
| 5 | Combination (10) | 1 | 3 | |

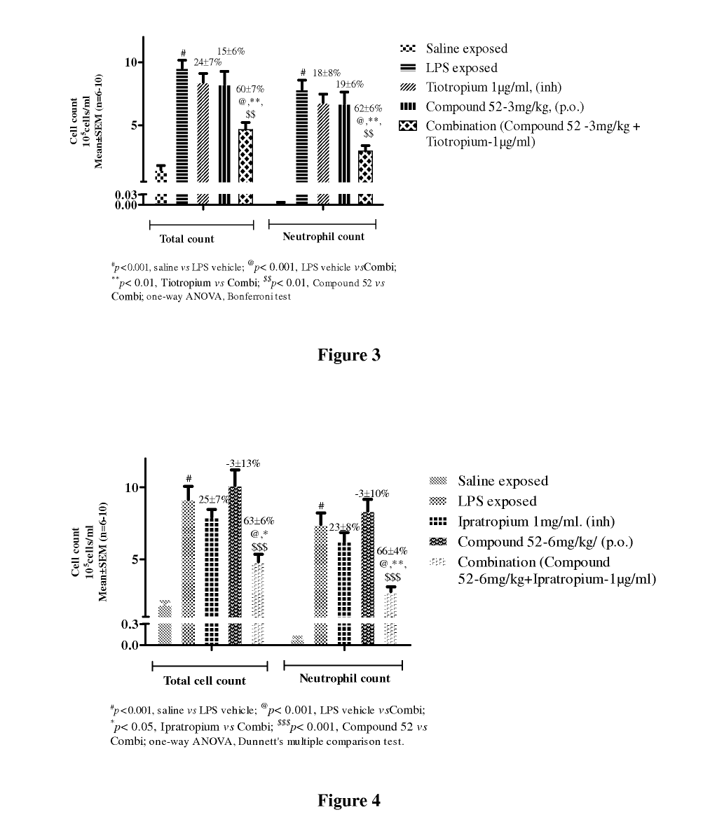

Combination of Compound 52 with tiotropium showed significant inhibition in LPS induced neutrophilia compared to the individual treatments (Table 7 and FIG. 3). Compound 52 in combination with tiotropium showed synergy in inhibition of neutrophilia in LPS model in SD rats.

TABLE 7

| | | Dose | | No of neutrophils |
|---|---|---|---|---|
| | | Tiotropium | Compound 52 | (% inhibition |
| Groups | Treatment (n) | (mcg/ml, inh) | (mg/kg, p.o.) | mean ± S.E.M.) |
| 1 | Saline (6) | — | — | 0.0 ± 0.0 |
| 2 | LPS (8) | — | — | 7.9 ± 0.7^{#} |
| 3 | Compound 52 (8) | — | 3 | 6.7 ± 0.9 (17.9 ± 7.6) |
| 4 | Tiotropium (8) | 1 | — | 6.8 ± 0.7 (18.9 ± 5.6) |
| 5 | Combination (10) | 1 | 3 | 3.0 ± 0.4 (62.4 ± 5.5) ^{@,}**^{,$$} |

^{#}p < 0.001, saline vs LPS vehicle;
^{@} p < 0.001, LPS vehicle vs Combi;
**p < 0.01, Tiotropium vs Combi;
^{$$}p < 0.01, Compound 52 vs Combi; one-way ANOVA, Bonferroni test.

### Example 5

### Animal Studies for the Combination of TRPA1 Antagonist and Ipratropium Bromide

The effect of Compound 52 and ipratropium bromide on LPS induced neutrophilia in male SD rats was evaluated. Animals were grouped as mentioned in Table 8.

TABLE 8

| | | Dose | | |
|---|---|---|---|---|
| | | Ipratropium | Compound 52 | |
| Groups | Treatment (n) | (mg/ml, inh) | (mg/kg, p.o.) | Exposure to |
| 1 | Saline (7) | — | — | Saline |
| 2 | LPS (5) | — | — | LPS |
| 3 | Compound 52 (6) | — | 6 | |
| 4 | Ipratropium | 1 | — | |
| | bromide(6) | | | |
| 5 | Combination (6) | 1 | 6 | |

LPS at a concentration of 100 mcg ml⁻¹ was nebulized for 40 min at 0.4 ml/min and a pressure of 1.7 psig in a Perspex exposure chamber (1.5×1×1 ft) fitted with nebulizer (RCI Hudson). Control animals were given saline exposure under similar conditions.

All the animals were treated as mentioned in Table 8. Compound 52 (6 mg/kg/5 ml) and ipratropium bromide (1 mg/ml as inhalation for 10 min at the rate of 0.3 ml/min) were administered 2 and 0 hour prior to LPS exposure respectively.

Bronchoalveolar lavage (BAL) was done after 4 h of LPS exposure. Animals were anesthetized with urethane (1.2 gm/kg/5 mL, i.p. in normal saline) and BAL was done with PBS (3 ml). This procedure was repeated three times and the BAL fluid was pooled for the measurement of total leukocyte.

Remaining BAL fluid was centrifuged at 4000 rpm for 20 min. The pellet formed in the bottom of the tube was used for the smear preparation for differential leukocyte count estimation. The differential leukocyte count was done using Leishman's stain.

The total number of neutrophils in each BAL sample was calculated using the formula:

$$Total No . of neutrophils ( in BAL ) = \frac{Total cell count \times {10}^{5} \text{/} mL \times \% neutrophils}{100}$$

% inhibition of neutrophils was calculated using the following formula:

$$\% Inhibition of neutrophils = \frac{Avg . {neutrophils}_{( LPS control )} - {neutrophils}_{( treatment )}}{Avg . {neutrophils}_{( LPS control )} - Avg . {neutrophils}_{( Saline control )}} \times 100$$

Statistical analysis was performed using One Way ANOVA followed by Dunnett's multiple comparisons with the help of Graph Pad Prism software. Statistical significance was set at p<0.05.

### Results:

Combination of compound 52 with ipratropium showed significant inhibition in LPS induced neutrophilia compared to the respective individual treatments (Table 9 and FIG. 4). Compound 52 in combination with ipratropium showed significant synergy in inhibition of neurophilia in LPS model in SD rats.

TABLE 9

| | | Dose | | No of neutrophils |
|---|---|---|---|---|
| | | Ipratropium | Compound 52 | (% inhibition; |
| Groups | Treatment (n) | (mg/ml, inh) | (mg/kg, p.o.) | mean ± S.E.M.) |
| 1 | Saline (6) | — | — | 0.02 ± 0.02 |
| 2 | LPS (5) | — | — | 8.1 ± 1.4* |
| 3 | Compound 52 (6) | — | 6 | 8.4 ± 0.8 (−3.2 ± 10.0) |
| 4 | Ipratropium (6) | 1 | — | 6.2 ± 0.6 (23.0 ± 7.6) |
| 5 | Combination (6) | 1 | 6 | 2.8 ± 0.3 (66.1 ± 3.9) ^{@,}**^{,$$$} |

^{#}p < 0.001, saline vs LPS vehicle;
^{@} p < 0.001, LPS vehicle vsCombi;
*p < 0.05, Ipratropium vs Combi;
^{$$$}p < 0.001, Compound 52 vs Combi; one-way ANOVA, Dunnett's multiple comparison test.

Although the invention herein has been described with reference to particular embodiments, it is to be understood that these embodiments are merely illustrative of the principles and application of the present invention. It is therefore to be understood that numerous modifications may be made to the illustrative embodiments of the present invention as described.

All publications, patents, and patent applications cited in this application are herein incorporated by reference to the same extent as if each individual publication, patent, or patent application was specifically and individually indicated to be incorporated herein by reference.

1-25. (canceled)

26. A pharmaceutical composition comprising synergistically effective amount of a TRPA1 antagonist that has an IC₅₀ for inhibiting human TRPA1 receptor activity of less than 1 micromolar having structure of formulae: (XII) or (D) 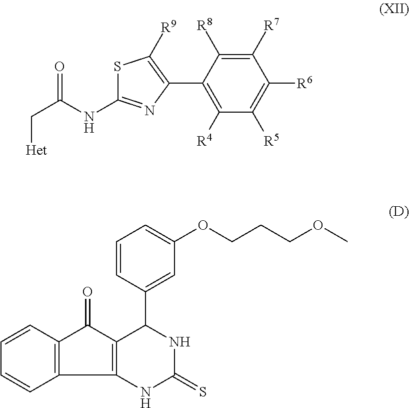
or a pharmaceutically-acceptable salt thereof, wherein,
‘Het’ is selected from the group consisting of 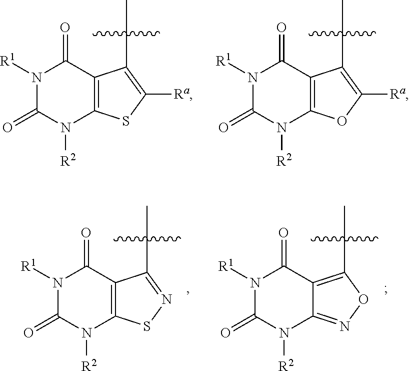
R¹, R² and R^{a}, which may be the same or different, are each independently hydrogen or (C₁-C₄)alkyl;
R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl;
and an anticholinergic agent.

27. The pharmaceutical composition according to claim 26, wherein the anticholinergic agent comprises tiotropium, oxitropium, ipratropium, glycopyrrolate and aclidinium or a salt thereof.

28. The pharmaceutical composition according to claim 26, wherein the TRPA1 antagonist and the anticholinergic agent are present in a weight ratio from about 1:0.0001 to about 1:10000.

29. A pharmaceutical composition comprising synergistically effective amount of a TRPA1 antagonist having structure of formula: 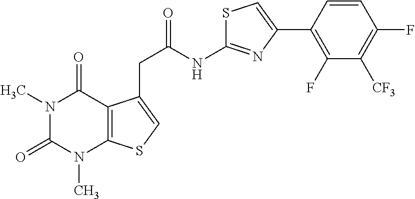 [SMILES: Cn1c(=O)c2c(CC(=O)Nc3nc(-c4ccc(F)c(C(F)(F)F)c4F)cs3)csc2n(C)c1=O]
and an anticholinergic agent.

30. The pharmaceutical composition according to claim 29, wherein the anticholinergic agent comprises tiotropium, oxitropium, ipratropium, glycopyrrolate and aclidinium or a salt thereof.

31. The pharmaceutical composition according to claim 29, wherein the composition is for inhalation administration, and the TRPA1 antagonist and the anticholinergic agent are present in a weight ratio from about 1:0.001 to about 1:300.

32. The pharmaceutical composition according to claim 29, wherein the composition is a fixed dose combination.

33. A pharmaceutical composition for inhalation administration comprising a TRPA1 antagonist having structure of formula: 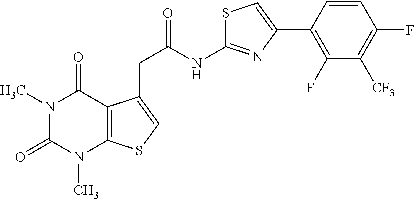 [SMILES: Cn1c(=O)c2c(CC(=O)Nc3nc(-c4ccc(F)c(C(F)(F)F)c4F)cs3)csc2n(C)c1=O]
and an anticholinergic agent selected from the group consisting of tiotropium, oxitropium, ipratropium, glycopyrrolate and aclidinium or a salt thereof.

34. The pharmaceutical composition according to claim 33, wherein the TRPA1 antagonist and the anticholinergic agent are present in a weight ratio from about 1:0.001 to about 1:300.

35. The pharmaceutical composition according to claim 33, wherein the composition is a fixed dose combination.

36. A method of treating a respiratory disorder in a subject, said method comprising administering to the subject the pharmaceutical composition according to claim 26.

37. A method of treating a respiratory disorder in a subject, said method comprising administering to the subject the pharmaceutical composition according to claim 29.

38. A method of treating a respiratory disorder in a subject, said method comprising administering to the subject the pharmaceutical composition according to claim 33.

39. A method of treating asthma or COPD in a subject, said method comprising administering to the subject the pharmaceutical composition according to claim 29.

40. A method of treating asthma or COPD in a subject, said method comprising administering to the subject the pharmaceutical composition according to claim 33.
